(19) United States
(12) Patent Application Publication
(10) Pub. No.: US 2002/0090649 A1
(43) Pub. Date: Jul. 11, 2002

(54) High density column and row addressable electrode arrays

(76) Inventors: Tony Chan, Scottsdale, AZ (US); Vi-En Choong, Chandler, AZ (US); Changming Li, Phoenix, AZ (US); George Nicolas Maracas, Phoenix, AZ (US); Larry Akio Nagahara, Phoenix, AZ (US); Song Shi, Phoenix, AZ (US)

Correspondence Address: FLEHR HOHBACH TEST ALBRITTON & HERBERT LLP, Suite 3400, Four Embarcadero Center, San Francisco, CA, 94111-4187, US

(21) Appl. No.: 09/945,154

(22) Filed: Aug. 31, 2001

Related U.S. Application Data
(63) Continuation of application No. 09/652,284, filed on Aug. 31, 2000.
(63) Continuation of application No. 09/464,500, filed on Dec. 15, 1999.
(60) Provisional application No. 60/299,780, filed on Jun. 20, 2001.

(51) Int. Cl. G01N 33/53; C12M 1/34; G01F 1/64

(52) U.S. Cl. 435/7.1; 435/287.2; 205/777.5

(57) ABSTRACT

This invention relates to the detection of biomolecules. Specifically, the invention relates to electronic or electrochemical detection of biomolecules using biochip arrays. In particular, the invention provides an apparatus comprising a platform for a column-and-row addressable, high-density, enhanced-sensitivity biochip array, and methods of use thereof. The devices and methods of the invention can be used to detect molecular interactions such as nucleic acid hybridization or protein binding.

This application is a continuing application of U.S. Ser. Nos. 09/652,284, filed Aug. 31, 2000 and 09/464,500, filed Dec. 15, 1999.

### FIELD OF THE INVENTION

This invention relates to the detection of biomolecules. Specifically, the invention relates to electronic or electrochemical detection of biomolecules using biochip arrays. In particular, the invention provides an apparatus comprising a platform for a column-and-row addressable, high-density, enhanced-sensitivity biochip array, and methods of use thereof. The devices and methods of the invention can be used to detect molecular interactions such as nucleic acid hybridization or protein binding.

### BACKGROUND OF THE INVENTION

A number of commonly utilized biological applications, including diagnoses of genetic disease, sequence-polymorphisms, analyses of gene expression, and studies of receptor-ligand interactions, rely on the ability to readily detect events related to probe-target interactions. In the past decades, autoradiography and fluorescence detection technologies have been used extensively in the molecular detection area.

Methods for electrical or electrochemical detection of molecular interactions between biomolecules have provided an attractive alternative to detection techniques relying on radioactive or fluorescent labels. Electrical and electrochemical detection techniques are based on the detection of alterations in the electrical properties of an electrode arising from interactions between one group of molecules attached to the surface of an electrode (often referred to as “probe” molecules) and another set of molecules present in a reaction mixture (often referred to as “target” molecules) contacted with the electrode. Methods and devices related to electrical or electrochemical detection of biomolecules are disclosed in U.S. Pat. Nos. 4,072,576, 4,098,645, 4,414,323, 4,840,893, 5,164,319, 5,187,096, 5,194,133, 5,312,527, 5,532,128, 5,591,578, 5,653,939, 5,670,322, 5,705,348, 5,770,369, 5,780,234, 5-,824,473, 5,891,630, 6,017,696 and International Application, Pub. No. WO 97/01646.

Electrical or electrochemical detection eliminates many of the disadvantages inherent in use of radioactive or fluorescent labels to detect interactions between the probe and target molecules. This process offers, for example, a detection technique that is safe, inexpensive, and sensitive, and is not burdened with complex and onerous regulatory requirements.

The development of microfabricated arrays (microarrays) of biomolecules has led to further improvements on traditional analytical techniques for the detection of molecular interactions between biomolecules. Microarrays of biomolecules (e.g., oligonucleotides, nucleic acids, proteins, peptides, or antibodies) have utility in a wide variety of applications in which molecular interactions between target molecules in a reaction mixture and large numbers of distinct probe molecules bound to defined regions of a substrate can be simultaneously assayed using electrical, optical, or radioactive detection strategies. Microarrays, therefore, satisfy the demand for inexpensive, high-throughput detection of biomolecular interactions.

Although biochip arrays for the electrochemical detection of molecular interactions between biomolecules have been proposed in the prior art, these devices have significant disadvantages. For example, the device disclosed by Egger et al. in U.S. Pat. Nos. 5,670,322 and 5,532,128 cannot be made column-and-row (or “x-y”) addressable, thus limiting the density of the test sites in the array and the usefulness of the apparatus. In U.S. Pat. No. 5,653,939, Hollis et al. disclose an x-y addressable array wherein a solid supporting substrate comprises a plurality of test sites in electrochemical contact with a set of orthogonally oriented electrodes. However, Hollis et al. does not provide an apparatus for efficient electrochemical detection of molecular interactions on porous, polymeric pads. Furthermore, Hollis et al. does not provide an apparatus having interdigitated electrodes.

These electrochemical methods can be based on a variety of mechanisms, as outlined herein and in the cited art. In some systems, the target analyte is selectively modified at specific sites with redox active moieties such as transition metal complexes, which can then be detected upon binding, using a variety of systems. Another approach is intercalation of redox-active moieties, e.g. into duplex DNA strands. Additionally, methods of detecting nucleic acids using oxidization of guanine bases has been described in U.S. Pat. Nos. 6,180,346; 6,127,127; 5,968,745 and 5,871,918.

However, there remains a need in the art to develop more efficient devices and methods for the detection of molecular interactions between biomolecules. In particular, there remains a need in the art for more efficient devices and methods for the electrical or electrochemical detection of molecular interactions. More particularly, there remains a need in the art to develop column and-row addressable biochip arrays for the electrical or electrochemical detection of molecular interactions that can be easily and cost-effectively fabricated, and that reduce the cost of performing various analyses, while increasing the effectiveness and utility thereof. The development of such devices, and methods for their use, would have wide application in the medical, genetic, and molecular biological arts.

### SUMMARY OF THE INVENTION

In accordance with the objects outlined above, the present invention provides apparatus for the detection of target analytes comprising a solid support comprising a grid of electrodes. The support comprises a first channel comprising a first row of first electrodes each comprising capture binding ligands and at least a second channel comprising a second row of second electrodes comprising capture binding ligands. The first electrodes share a first row interconnect, the second electrodes share a second row interconnect, and each column of corresponding first and second electrodes share a column interconnect.

In a further aspect, the invention provides apparatus for the detection of target analytes comprising a solid support comprising a grid array of wells, each well comprising a first electrode comprising first capture binding ligands, an insulating layer, and at least a second electrode comprising second capture binding ligands.

In a further aspect, the invention comprises methods for detecting a target analyte comprising contacting a sample comprising a target analyte with a apparatus comprising a solid support comprising a grid of electrodes. The support comprises a first channel comprising a first row of first electrodes each comprising capture binding ligands and at least a second channel comprising a second row of second electrodes comprising capture binding ligands. The first electrodes share a first row interconnect, the second electrodes share a second row interconnect, and each column of corresponding first and second electrodes share a column interconnect. The method is carried out under conditions wherein the target analyte will bind at least one of the capture binding ligands, and the target analyte is detected.

In an additional aspect, the invention provides methods for detecting a target analyte comprising contacting a sample comprising a target analyte with a apparatus comprising a solid support comprising a grid array of wells. Each well comprises a first electrode comprising first capture binding ligands, an insulating layer, and at least a second electrode comprising second capture binding ligands. The assay is done under conditions wherein the target analyte will bind at least one of the capture binding ligands, and the target analyte is detected.

One apparatus of the present invention comprises a supporting substrate comprising an array of test sites; a plurality of porous, polymeric pads in contact with the supporting substrate at the test sites; a set of input electrodes in contact with the plurality of porous, polymeric pads at the test sites, wherein each input electrode is arranged to address a subset of the test sites; a set of output electrodes in contact with the plurality of porous, polymeric pads at the test sites, wherein each output electrode is arranged to address a subset of the test sites, and wherein each output electrode is in electrochemical contact with an input electrode; a plurality of linker moieties in contact with the porous, polymeric pads at the test sites; a plurality of probe molecules immobilized to the linker moieties, wherein said probe molecules specifically bind to or interact with target molecules; a means for producing an electrical signal at each input electrode; a means for detecting changes in the electrical signal at each output electrode; and an electrolyte solution in contact with the porous polymeric pads, input electrodes, output electrodes, linker moieties, and probe molecules.

Another apparatus of the present invention comprises a supporting substrate comprising an array of test sites; a plurality of porous, polymeric pads in contact with the supporting substrate at the test sites; a set of input electrodes in contact with the plurality of porous, polymeric pads at the test sites, wherein each input electrode is arranged to address a subset of the test sites; a set of output electrodes in contact with the plurality of porous, polymeric pads at the test sites, wherein each output electrode is arranged to address a subset of the test sites, and wherein each output electrode is in electrochemical contact with an input electrode; a plurality of linker moieties in contact with the porous, polymeric pads at the test sites; a plurality of probe molecules immobilized to the linker moieties, wherein said probe molecules specifically bind to or interact with target molecules; at least one reference electrode in electrochemical contact with the input and output electrodes; a means for producing an electrical signal at each input electrode; a means for detecting changes in the electrical signal at each output electrode; and an electrolyte solution in contact with the porous polymeric pads, input electrodes, output electrodes, linker moieties, and probe molecules.

Still another apparatus of the present invention comprises a supporting substrate comprising an array of test sites; a set of input electrodes in contact with the supporting substrate, wherein each input electrode is arranged to address a subset of the test sites; a set of output electrodes in contact with the supporting substrate at the test sites, wherein each output electrode is arranged to address a subset of the test sites, each output electrode is in electrochemical contact with an input electrode, and the output electrodes and input electrodes are interdigitated at the test site; a plurality of linker moieties in contact with either the input electrodes, the output electrodes, or both the input electrodes and output electrodes at the test sites; a plurality of probe molecules immobilized to the linker moieties, wherein said probe molecules specifically bind to or interact with target molecules; a means for producing an electrical signal at each input electrode; a means for detecting changes in the electrical signal at each output electrode; and an electrolyte solution in contact with the input electrodes, output electrodes, linker moieties, and probe molecules.

Still another apparatus of the present invention comprises a supporting substrate comprising an array of test sites; a set of input electrodes in contact with the supporting substrate, wherein each input electrode is arranged to address a subset of the test sites; a set of output electrodes in contact with the supporting substrate at the test sites, wherein each output electrode is arranged to address a subset of the test sites, each output electrode is in electrochemical contact with an input electrode, and the output electrodes and input electrodes are interdigitated at the test site; a plurality of linker moieties in contact with either the input electrodes, the output electrodes, or both the input electrodes and output electrodes at the test sites; a plurality of probe molecules immobilized to the linker moieties, wherein said probe molecules specifically bind to or interact with target molecules; at least one reference electrode in electrochemical contact with the input and output electrodes; a means for producing an electrical signal at each input electrode; a means for detecting changes in the electrical signal at each output electrode; and an electrolyte solution in contact with the input electrodes, output electrodes, linker moieties, reference electrode, and probe molecules.

The apparatus of the present invention may further comprise a plurality of wells wherein each well encompasses a porous, polymeric pad, wherein a plurality of probe molecules is immobilized to linker moieties that are in contact with the porous, polymeric pad; an input electrode, and an output electrode. Preferably, the probe molecules in any particular well are identical to each other, while each well comprises probe molecules unique to that well.

The present invention provides methods employing the apparatus that are useful for electrical or electrochemical detection of molecular interactions between probe molecules immobilized to linker moieties in contact with porous, polymeric pads and target molecules in a sample solution. In one method of the present invention, a first electrical signal is applied at an input electrode in contact with a first set of porous, polymeric pads, wherein the first set of porous, polymeric pads comprises the porous, polymeric pad at the specific test site; and the first electrical signal is then detected at an output electrode in contact with a second set of porous, polymeric pads, wherein the second set of porous, polymeric pads comprises the porous, polymeric pad at the specific test site. Thereafter, the first and second sets of porous, polymeric pads are exposed to a sample mixture containing a particular target molecule; a second electrical signal is applied at an input electrode in contact with the first set of porous, polymeric pads; and the second electrical signal is detected at an output electrode in contact with the second set of porous, polymeric pads. The first and second electrical signals are compared, and molecular interactions between immobilized probe molecules and target molecules in the sample mixture are detected by determining that the first electrical signal is different from the second electrical signal.

In some embodiments of the methods of the present invention, target molecules in a sample mixture are labeled with an electrochemically-active reporter molecule prior to exposing the first and second sets of porous, polymeric pads to the sample mixture.

The x-y addressable bioarrays of the present invention can be employed for both electrical and electrochemical detection, thus permitting a wider number of analyses to be performed on these devices. The x-y addressing scheme simplifies and reduces the number of electrode interconnections required, thus permitting the bioarrays of the present invention to be more cost-effectively fabricated. The three-dimensional design of the input and output electrodes increases the surface area of the electrodes, thereby increasing the efficiency by which the devices can be used to electrically and electrochemically detect molecular interactions between biomolecules. Furthermore, in those devices of the invention in which the input and output electrodes are interdigitated, such interdigitation allows one with skill in the art to fabricate a more efficient device for a particular electrical or electrochemical detection scheme by altering the distance between the input and output electrodes.

### BRIEF DESCRIPTION OF THE DRAWINGS

FIG. 1 illustrates a schematic representation of a cross-section view of the device platform.

FIG. 2 illustrates a schematic representation of a top view of the device platform.

FIG. 3 is a schematic diagram of the row/column configuration of a high-density array useful in the practice of the invention.

FIG. 4 is a schematic diagram of the row/column configuration of a high density array using common interconnects and electrodes.

FIG. 5 illustrates a schematic representation of a top view of one embodiment of the x-y addressable microarray of the present invention. The microarray comprises a supporting substrate 1 comprising a plurality of optional porous, polymeric pads 2, the porous, polymeric pads defining an array of test sites. A set of input electrodes 3 is fabricated within or on top of the supporting substrate 1, the set of input electrodes 3 being arranged so that each input electrode addresses a subset of test sites. A set of output electrodes 4 is fabricated within or on top of the supporting substrate 1, the set of output electrodes 4 being arranged so that each output electrode 4 addresses a subset of test sites. In this embodiment, the input electrodes 3 and the output electrodes 4 are embedded within the porous, polymeric pads 2 and the input electrodes 3 and the output electrodes 4 are optionally arranged so that they interdigitate.

FIGS. 6A and 6B illustrate schematic representations of two cross-section views of one embodiment of the x-y addressable microarray of the present invention.

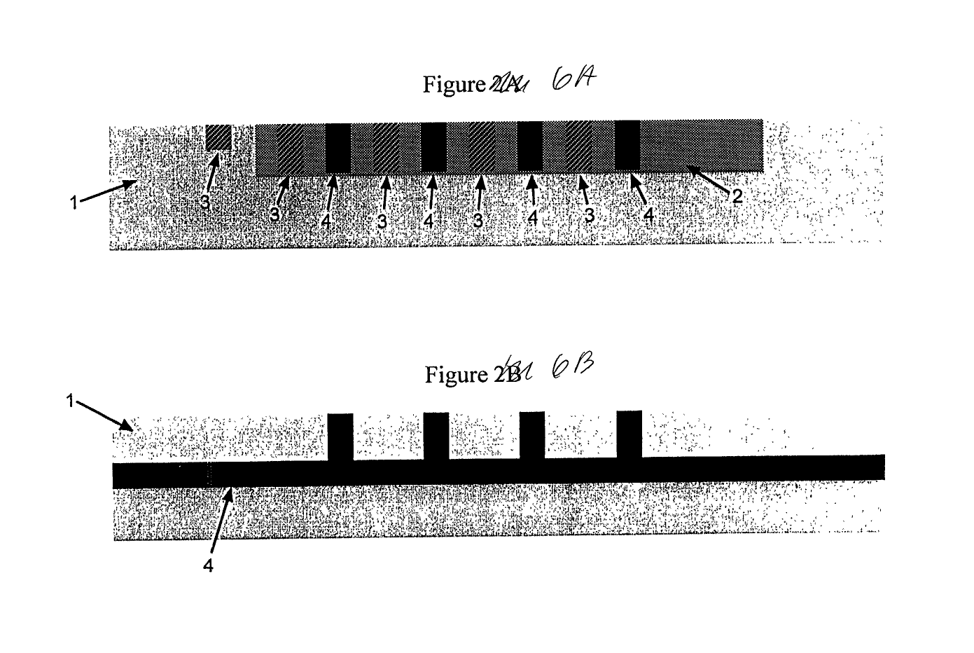

FIG. 7 illustrates a schematic representation of a top view of another embodiment of the x-y addressable microarray of the present invention. The microarray comprises a supporting substrate 1 comprising a plurality of porous, polymeric pads 2, the porous, polymeric pads defining an array of test sites. A set of input electrodes 3 is fabricated within or on top of the supporting substrate 1, the set of input electrodes 3 being arranged so that each input electrode 3 addresses a subset of test sites. A set of output electrodes 4 is fabricated within or on top of the supporting substrate 1, the set of output electrodes 4 being arranged so that each output electrode 4 addresses a subset of test sites. In this embodiment, the input electrodes 3 and the output electrodes 4 are embedded within the porous, polymeric pads 2 and the input electrodes 3 and the output electrodes 4 are arranged so that they interdigitate (see FIG. 5). A reference electrode 5 is separated from the input electrodes 3 and output electrodes 4 by either a portion of the supporting substrate 1 (or optionally by an additional insulating layer). Electrochemical contact between the reference electrode S and the input electrodes 3 and output electrodes 4 is established through a via 6 fabricated at each test site.

FIGS. 8A and 8B illustrate schematic representations of two cross-section views of one embodiment of the x-y addressable microarray of the present invention. The microarrays illustrated in the Figures can be used for the electrical or electrochemical detection of molecular interactions between biomolecules.

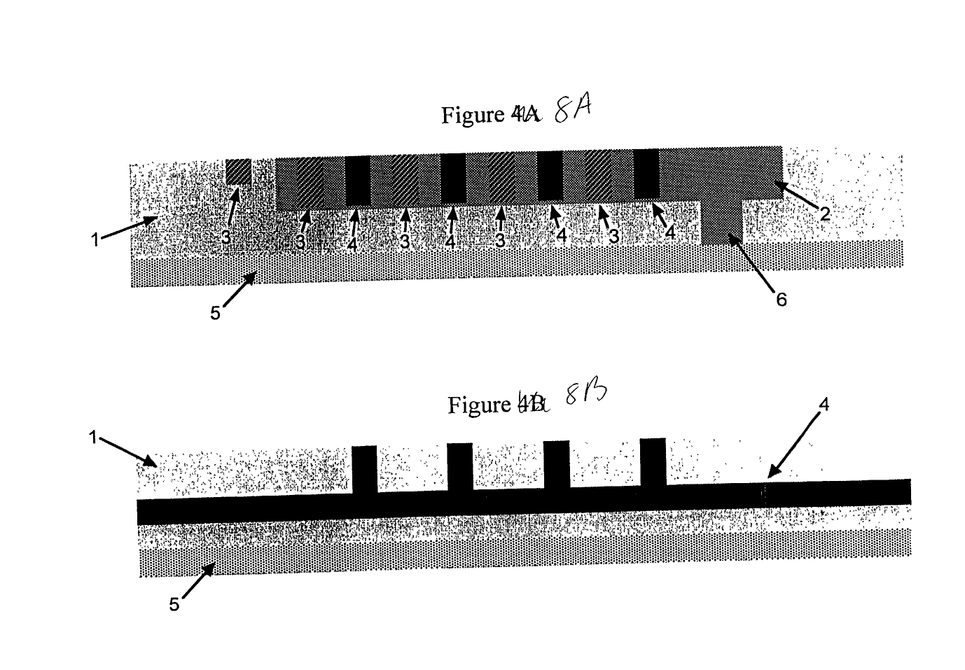

FIGS. 9A, B, C and D depict a variety of different embodiments of the invention. FIGS. 9A, 9B and 9C depict the use of microchannels, and FIG. 9A depicts the use of the channels in rows with a top; FIG. 9C depicts one configuration, FIG. 9D a different embodiment, with a hydrophobic insulation layer. FIG. 9B depicts the two dimensional device without microchannels.

FIG. 10 depicts the use of a valve, in this case an air bubble, to ionically isolate two channels.

FIGS. 11A and 11B depict several embodiments of the three dimensional addressable array. FIG. 11B utilizes a hydrophobic insulation layer to keep the wells fluidically separated, but a top could be used as well.

### DETAILED DESCRIPTION OF THE INVENTION

The present invention is directed to a variety of devices (sometimes referred to herein as “biochips) comprising arrays of electrodes. As outlined above, electronic detection of biomolecules such as nucleic acids and proteins provides a number of commercial advantages. However, due to the fact that electronic detection methods rely on electrodes requiring interconnects, generally one or two per electrode depending on the method of detection, achieving the high density required in some commercial applications with electrode arrays can be problematic. Accordingly, the present invention provides a variety of methods of achieving higher density of electrode arrays by using common interconnects in an X-Y addressable mode, such that rows and columns of electrodes share interconnects or leads. For example, a two dimensional 100 by 100 array requiring two interconnects at each electrode would normally require 20,000 leads, but using the devices and methods of the invention 200 leads can be used, by having electrodes in each row share an interconnect and electrodes in each column share a different interconnect. Thus by activating one row interconnect and one column interconnect, a single electrode can be addressed. In one embodiment, each electrode is contained within a single well, to prevent sample cross-over. In addition, by adding microchannels that encompass rows of electrodes, the electrodes are both ionically isolated for the purposes of detection, and sample handling and multiplexing is facilitated. These microchannels can be either open, with hydrophobic material surrounding them to prevent fluidic “short circuiting”, using any number of known hydrophobic materials, including plastics and other polymers, waxes, greases, etc. Alternatively, the channels can be closed, using a sealing top to the channel, made out any number of different materials, including the flexible tops outlined in PCT US01/02664, or of any other material outlined herein for substrates. In addition, sealing gaskets of rubber or silicone or other suitable material may be used. The same is true when individual wells are used.

In addition, the invention provides multilayered electrode arrays, which are made by building up layers of these two dimensional arrays. In this embodiment, the electrode arrays are placed in a grid format that comprises individual wells, similar to a microtiter plate. Each well comprises a plurality of electrodes, placed in the Z direction, separated by insulating material. A particular advantage is that all Z dimension electrodes can share a counter/reference electrode. This can further reduce the number of connection wires. If the number of the layer of an N×N array is M, (2M−1)×N² test sites only need 2×M×N interconnects to provide the necessary connections (or M×N for embodiments that do not require two electrodes at each test site).

Accordingly, the present invention provides compositions and methods for detecting the presence or absence of target analytes in samples. As will be appreciated by those in the art, the sample solution may comprise any number of things, including, but not limited to, bodily fluids (including, but not limited to, blood, urine, serum, lymph, saliva, anal and vaginal secretions, perspiration and semen, of virtually any organism, with mammalian samples being preferred and human samples being particularly preferred); environmental samples (including, but not limited to, air, agricultural, water and soil samples); biological warfare agent samples; research samples (i.e. in the case of nucleic acids, the sample may be the products of an amplification reaction, including both target and signal amplification as is generally described in PCT/US99/01705, such as PCR amplification reaction); purified samples, such as purified genomic DNA, RNA, proteins, etc.; raw samples (bacteria, virus, genomic DNA, etc.); as will be appreciated by those in the art, virtually any experimental manipulation may have been done on the sample.

The methods are directed to the detection of target analytes. By “target analyte” or “analyte” or grammatical equivalents herein is meant any molecule or compound to be detected and that can bind to a binding species, defined below. Suitable analytes include, but not limited to, small chemical molecules such as environmental or clinical chemical or pollutant or biomolecule, including, but not limited to, pesticides, insecticides, toxins, therapeutic and abused drugs, hormones, antibiotics, antibodies, organic materials, etc. Suitable biomolecules include, but are not limited to, proteins (including enzymes, immunoglobulins and glycoproteins), nucleic acids, lipids, lectins, carbohydrates, hormones, whole cells (including procaryotic (such as pathogenic bacteria) and eucaryotic cells, including mammalian tumor cells), viruses, spores, etc. Particularly preferred analytes are proteins including enzymes; drugs, cells; antibodies; antigens; cellular membrane antigens and receptors (neural, hormonal, nutrient, and cell surface receptors) or their ligands.

In a preferred embodiment, the target analyte is a protein. As will be appreciated by those in the art, there are a large number of possible proteinaceous target analytes that may be detected using the present invention. By “proteins” or grammatical equivalents herein is meant proteins, oligopeptides and peptides, derivatives and analogs, including proteins containing non-naturally occurring amino acids and amino acid analogs, and peptidomimetic structures. The side chains may be in either the (R) or the (S) configuration. In a preferred embodiment, the amino acids are in the (S) or L-configuration. As discussed below, when the protein is used as a binding ligand, it may be desirable to utilize protein analogs to retard degradation by sample contaminants.

Suitable protein target analytes include, but are not limited to, (1) immunoglobulins, particularly IgEs, IgGs and IgMs, and particularly therapeutically or diagnostically relevant antibodies, including but not limited to, for example, antibodies to human albumin, apolipoproteins (including apolipoprotein E), human chorionic gonadotropin, cortisol, α-fetoprotein, thyroxin, thyroid stimulating hormone (TSH), antithrombin, antibodies to pharmaceuticals (including antieptileptic drugs (phenytoin, primidone, carbariezepin, ethosuximide, valproic acid, and phenobarbitol), cardioactive drugs (digoxin, lidocaine, procainamide, and disopyramide), bronchodilators ( theophylline), antibiotics (chloramphenicol, sulfonamides), antidepressants, immunosuppresants, abused drugs (amphetamine, methamphetamine, cannabinoids, cocaine and opiates) and antibodies to any number of viruses or bacteria outlined below.

As will be appreciated by those in the art, a large number of analytes may be detected using the present methods; basically, any target analyte for which a binding ligand, described below, may be made may be detected using the methods of the invention.

In a preferred embodiment, the target analytes are nucleic acids. By “nucleic acid” or “oligonucleotide” or grammatical equivalents herein means at least two nucleotides covalently linked together. A nucleic acid of the present invention will generally contain phosphodiester bonds, although in some cases, as outlined below, nucleic acid analogs are included that may have alternate backbones, comprising, for example, phosphoramide (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphophoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with bicyclic structures including locked nucleic acids, Koshkin et al., J. Am. Chem. Soc. 120:13252-3 (1998); positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within the definition of nucleic acids (see Jenkins et al., Chem. Soc. Rev. (1995) pp169-176). Several nucleic acid analogs are described in Rawls, C & E News Jun. 2, 1997 page 35. All of these references are hereby expressly incorporated by reference. These modifications of the ribose-phosphate backbone may be done to facilitate the addition of ETMs, or to increase the stability and half-life of such molecules in physiological environments.

As will be appreciated by those in the art, all of these nucleic acid analogs may find use in the present invention. In addition, mixtures of naturally occurring nucleic acids and analogs can be made. Alternatively, mixtures of different nucleic acid analogs, and mixtures of naturally occurring nucleic acids and analogs may be made.

Particularly preferred in some embodiments are peptide nucleic acids (PNA) which includes peptide nucleic acid analogs. These backbones are substantially non-ionic under neutral conditions, in contrast to the highly charged phosphodiester backbone of naturally occurring nucleic acids. This results in two advantages. First, the PNA backbone exhibits improved hybridization kinetics. PNAs have larger changes in the melting temperature (Tm) for mismatched versus perfectly matched basepairs. DNA and RNA typically exhibit a 2-4° C. drop in Tm for an internal mismatch. With the non-ionic PNA backbone, the drop is closer to 7-9° C. Similarly, due to their non-ionic nature, hybridization of the bases attached to these backbones is relatively insensitive to salt concentration. This can be advantageous in the systems of the present invention, as a reduced salt hybridization solution has a lower Faradaic current than a physiological salt solution (in the range of 150 mM).

The nucleic acids may be single stranded or double stranded, as specified, or contain portions of both double stranded or single stranded sequence. The nucleic acid may be DNA, both genomic and cDNA, RNA or a hybrid, where the nucleic acid contains any combination of deoxyribo- and ribo-nucleotides, and any combination of bases, including uracil, adenine, thymine, cytosine, guanine, inosine, xathanine hypoxathanine, isocytosine, isoguanine, etc. A preferred embodiment utilizes isocytosine and isoguanine in nucleic acids designed to be complementary to other probes, rather than target sequences, as this reduces non-specific hybridization, as is generally described in U.S. Pat. No. 5,681,702. As used herein, the term “nucleoside” includes nucleotides as well as nucleoside and nucleotide analogs, and modified nucleosides such as amino modified nucleosides. In addition, “nucleoside” includes non-naturally occurring analog structures. Thus for example the individual units of a peptide nucleic acid, each containing a base, are referred to herein as a nucleoside.

Thus, in a preferred embodiment, the target analyte is a target sequence. The term “target sequence” or “target nucleic acid” or grammatical equivalents herein means a nucleic acid sequence on a single strand of nucleic acid. The target sequence may be a portion of a gene, a regulatory sequence, genomic DNA, cDNA, RNA including mRNA and rRNA, or others. As is outlined herein, the target sequence may be a target sequence from a sample, or a secondary target such as a product of an amplification reaction, etc. It may be any length, with the understanding that longer sequences are more specific. As will be appreciated by those in the art, the complementary target sequence may take many forms. For example, it may be contained within a larger nucleic acid sequence, i.e. all or part of a gene or mRNA, a restriction fragment of a plasmid or genomic DNA, among others. As is outlined more fully below, probes are made to hybridize to target sequences to determine the presence or absence of the target sequence in a sample. Generally speaking, this term will be understood by those skilled in the art. The target sequence may also be comprised of different target domains; for example, a first target domain of the sample target sequence may hybridize to a capture probe and a second target domain may hybridize to a portion of an amplifier probe, a label probe, or a different capture or capture extender probe, etc. The target domains may be adjacent or separated as indicated. Unless specified, the terms “first” and “second” are not meant to confer an orientation of the sequences with respect to the 5′-3′ orientation of the target sequence. For example, assuming a 5′-3′ orientation of the complementary target sequence, the first target domain may be located either 5′ to the second domain, or 3′ to the second domain.

Suitable target analytes include biomolecules associated with: (1) viruses, including but not limited to, orthomyxoviruses, (e.g. influenza virus), paramyxoviruses (e.g respiratory syncytial virus, mumps virus, measles virus), adenoviruses, rhinoviruses, coronaviruses, reoviruses, togaviruses (e.g. rubella virus), parvoviruses, poxviruses (e.g. variola virus, vaccinia virus), enteroviruses (e.g. poliovirus, coxsackievirus), hepatitis viruses (including A, B and C), herpesviruses (e.g. Herpes simplex virus, varicella-zoster virus, cytomegalovirus, Epstein-Barr virus), rotaviruses, Norwalk viruses, hantavirus, arenavirus, rhabdovirus (e.g. rabies virus), retroviruses (including HIV, HTLV-I and -II), papovaviruses (e.g. papillomavirus), polyomaviruses, and picornaviruses, and the like; and (2) bacteria, including but not limited to, a wide variety of pathogenic and non-pathogenic prokaryotes of interest including Bacillus; Vibrio, e.g. V. cholerae; Escherichia, e.g. Enterotoxigenic E. coli, Shigella, e.g. S. dysenteriae; Salmonella, e.g. S. typhi; Mycobacterium e.g. M. tuberculosis, M. leprae; Clostridium, e.g. C. botulinum, C. tetani, C. difficile, C.perfringens; Cornyebacterium, e.g. C. diphtheriae; Streptococcus, S. pyogenes, S. pneumoniae; Staphylococcus, e.g. S. aureus; Haemophilus, e.g. H. influenzae; Neisseria, e.g. N. meningitidis, N. gonorrhoeae; Yersinia, e.g. G. lambliaY. pestis, Pseudomonas, e.g. P. aeruginosa, P. putida; Chlamydia, e.g. C. trachomatis; Bordetella, e.g. B. pertussis; Treponema, e.g. T. palladium; and the like.

Other suitable target analytes include, but are not limited to, (1) enzymes (and other proteins), including but not limited to, enzymes used as indicators of or treatment for heart disease, including creatine kinase, lactate dehydrogenase, aspartate amino transferase, troponin T, myoglobin, fibrinogen, cholesterol, triglycerides, thrombin, tissue plasminogen activator (tPA); pancreatic disease indicators including amylase, lipase, chymotrypsin and trypsin; liver function enzymes and proteins including cholinesterase, bilirubin, and alkaline phosphotase; aldolase, prostatic acid phosphatase, terminal deoxynucleotidyl transferase, and bacterial and viral enzymes such as HIV protease; (2) hormones and cytokines (many of which serve as ligands for cellular receptors) such as erythropoietin (EPO), thrombopoietin (TPO), the interleukins (including IL-1 through IL-17), insulin, insulin-like growth factors (including IGF-1 and -2), epidermal growth factor (EGF), transforming growth factors (including TGF-α and TGF-β), human growth hormone, transferrin, epidermal growth factor (EGF), low density lipoprotein, high density lipoprotein, leptin, VEGF, PDGF, ciliary neurotrophic factor, prolactin, adrenocorticotropic hormone (ACTH), calcitonin, human chorionic gonadotropin, cotrisol, estradiol, follicle stimulating hormone (FSH), thyroid-stimulating hormone (TSH), leutinzing hormone (LH), progeterone and testosterone; and (3) other proteins (including α-fetoprotein, carcinoembryonic antigen CEA, cancer markers, etc.).

Suitable target analytes include carbohydrates, including but not limited to, markers for breast cancer (CA15-3, CA 549, CA 27.29), mucin-like carcinoma associated antigen (MCA), ovarian cancer (CA125), pancreatic cancer (DE-PAN-2), prostate cancer (PSA), CEA, and colorectal and pancreatic cancer (CA 19, CA 50, CA242).

In a preferred embodiment, the methods of the invention are used to detect pathogens such as bacteria. In this embodiment, preferred target sequences include rRNA, as is generally described in U.S. Pat. Nos. 4,851,330; 5,288,611; 5,723,597; 6,641,632; 5,738,987; 5,830,654; 5,763,163; 5,738,989; 5,738,988; 5,723,597; 5,714,324; 5,582,975; 5,747,252; 5,567,587; 5,558,990; 5,622,827; 5,514,551; 5,501,951; 5,656,427; 5.352.579; 5,683,870; 5,374,718; 5,292,874; 5,780,219; 5,030,557; and 5,541,308, all of which are expressly incorporated by reference.

As will be appreciated by those in the art, a large number of analytes may be detected using the present methods; basically, any target analyte for which a binding ligand, described below, may be made may be detected using the methods of the invention. While many of the techniques described below exemplify nucleic acids as the target analyte, those of skill in the art will recognize that other target analytes can be detected using the same systems.

If required, the target analyte is prepared using known techniques. For example, the sample may be treated to lyse the cells, using known lysis buffers, electroporation, etc., with purification and/or amplification as needed, as will be appreciated by those in the art. When the target analyte is a nucleic acid, the target sequence may be amplified as required; suitable amplification techniques are outlined in PCT US99/01705, hereby expressly incorporated by reference. In addition, techniques to increase the amount or rate of hybridization can also be used; see for example WO 99/67425 and U.S. Ser. Nos. 09/440,371 and 60/171,981, all of which are hereby incorporated by reference.

The sample comprising the target analyte is added to a solid support comprising a grid of electrodes. By “substrate” or “solid support” or other grammatical equivalents herein is meant any material that can be modified to contain discrete individual sites (including wells) appropriate to the formation of electrodes and/or of the attachment or association of capture binding ligands. As outlined more fully below, the substrate may be a single material (for example in some embodiments of two dimensional arrays) or may be layers of materials (for example in three dimensional arrays). Suitable substrates include metal surfaces such as gold, electrodes as defined below, glass and modified or functionalized glass, fiberglass, teflon, ceramics, mica, plastic (including acrylics, polystyrene and copolymers of styrene and other materials, polypropylene, polyethylene, polybutylene, polyimide, polycarbonate, polyurethanes, Teflon™, and derivatives thereof, etc.), GETEK (a blend of polypropylene oxide and fiberglass), etc, polysaccharides, nylon or nitrocellulose, resins, silica or silica-based materials including silicon and modified silicon, carbon, metals, inorganic glasses and a variety of other polymers. In a preferred embodiment, the materials used to create the substrate are non-porous, although this is not required in some embodiments.

In a preferred embodiment, as more fully outlined below, the substrate comprises a number of different layers, including electrodes and insulating layers. By “insulating layer” herein is meant a layer of material that will not substantially transport electrons. Preferably, the insulating layer is a layer of insulative dielectric material, including, but not limited to, ceramics, plastics, printed circuit board materials, polymers, metal oxide or nitrides such as SiO₂, SiNₓ or A10_{x.}

Preferred substrates include ceramics, glass and printed circuit board (PCB) materials. Reference is made to U.S. Ser. No. 09/796,077; PCT US00/34145; PCT US01/02664; PCT/US00?33499; PCT/US00/33497; PCT/US99/23324; WO 01/34302; WO 98/20162; WO 98/112430; WO 00/16089; WO 99/57317; WO 99/67425; WO 01/35100; WO 00/62931 WO 01/06016; WO 01/07665; and PCT/US01/01150, all of which are expressly incorporated by reference in their entirety.

The present system finds particular utility in array formats, i.e. wherein there is a matrix of addressable detection electrodes (herein generally referred to “pads”, “addresses”, “test sites” or “micro-locations”). These test sites can have any convenient shape, e.g. circular, rectangular, elliptical or wedge-shaped. By “array” herein is meant a plurality of capture ligands in an array format; the size of the array will depend on the composition and end use of the array. Arrays containing from about 2 different capture ligands to many thousands can be made. Generally, the array will comprise from two to as many as 100,000 or more, depending on the size of the electrodes, as well as the end use of the array. Preferred ranges are from about 2 to about 10,000, with from about 5 to about 1000 being preferred, and from about 10 to about 100 being particularly preferred. In some embodiments, in some arrays, multiple substrates may be used, either of different or identical compositions. Thus for example, large arrays may comprise a plurality of smaller substrates.

In general, the electrodes of the invention are in a grid array format. By “grid” or “matrix” herein is meant that the individual test sites (e.g. electrodes) have precisely defined X and Y coordinates (and in some cases Z coordinates), so that a given electrode at a particular position in the array can be identified. In general, there are planar (two dimensional) grids, comprising a plurality of rows and columns. In addition, these planar grids can be stacked together to form three dimensional grids. In a preferred embodiment, the rows and columns of electrodes are at 90 degree angles to each other, to form a traditional X-Y grid, although as will be appreciated by those in the art, other geometries (trapezoids, etc.), can be exploited. What is important is that the electrodes in any particular row or column can share an interconnect or lead, even if this requires a circuitous route. In the three dimensional arrays of the invention, it is preferred that the rows and columns of each two dimensional array line up to allow the electrodes to share wells.

An embodiment of the present invention comprises a substrate with at least one surface comprising an array, and in a preferred embodiment, an array of electrodes. By “electrode” herein is meant a composition, which, when connected to an electronic device, is able to sense a current or charge and convert it to a signal. Alternatively an electrode can be defined as a composition which can apply a potential to and/or pass electrons to or from species in the solution. Preferred electodes are known in the art and include, but are not limited to, certain metals and their oxides, including gold; copper; silver; chromium; titanium; platinum; palladium; silicon; aluminum; metal oxide electrodes including platinum oxide, titanium oxide, tin oxide, indium tin oxide, palladium oxide, silicon oxide, aluminum oxide, molybdenum oxide (Mo₂O₆), ruthenium oxides, and zinc oxide and tungsten oxide (WO₃; both of which are transparent); conductive plastics (such as polymers like polythiophenes, polyanilines, polypyrroles, and metal impregnated polymers); and carbon (including glassy carbon electrodes, graphite and carbon paste). Preferred electrodes include gold, silicon, carbon and metal oxide electrodes, with gold being particularly preferred.

The electrodes described herein are depicted as a flat surface, which is only one of the possible conformations of the electrode and is for schematic purposes only.

The devices of the invention can take on a variety of different configurations. Device embodiments of the invention are useful for either electrical or electrochemical detection of interactions between biomolecules. As used herein, the term “electrochemical detection” is intended to encompass methods based on oxidation/reduction (redox) processes induced by electron transfer between electrodes, most preferably mediated by an electrochemical reporter group attached to the probe moiety, the target moiety, or both. In this embodiment, generally one electrode per test site is used, and the whole array can share a reference electrode.

In other embodiments, each test site requires two electrodes, for example in electrical detection methods. As used herein, the term “electrical detection” is intended to encompass methods that rely on impedance changes (such as resistance, capacitance and inductance) due to differences in electrical state occupancy in the biomolecules in the bound and unbound conformations.

In this embodiment, there may be input and output electrodes. As used herein, the term “input electrode” refers to an electrode that can be used to apply an electrical signal to a particular test site. In some embodiments, the electrical signal is applied to the input electrode using a multiplexer. As used herein, the term “multiplexer” refers to a device that allows electrical signals to be selectively applied to two or more input electrodes.

As used herein, the term “output electrode” refers to an electrode that can be used to detect an electrical signal at a particular test site. In some embodiments, the electrical signal is detected using a demultiplexer. As used herein, the term “demultiplexer” refers to a device that allows electrical signals from two or more output electrodes to be selectively detected at an electrical signal detection device.

As used herein, the term “reference electrode” refers to an electrode that can be used in assays where an estimate or determination of the number or concentration of target molecules in a sample solution is desired.

Accordingly, in a preferred embodiment, two dimensional arrays are provided, which can also be configured in a variety of ways. In a preferred embodiment, the two dimensional array comprises an X-Y addressable array of electrodes, wherein the rows and columns of electrodes share leads. In this embodiment, the electrodes preferably comprise either self-assembled monolayers or porous polymeric pads. Additionally, the two dimensional arrays (or in some cases, as is further outlined below, the three dimensional arrays) can comprise microchannels that feed rows of electrodes.

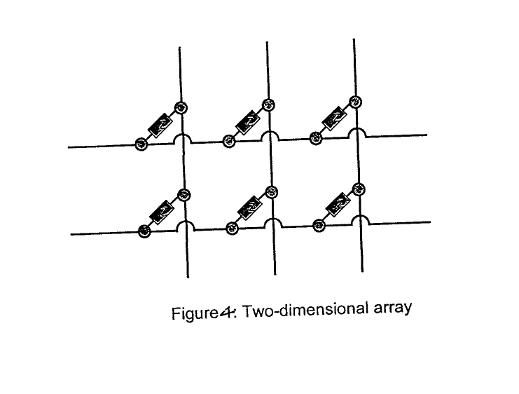

Accordingly, in a preferred embodiment, two dimensional arrays are provided with two electrodes at each test site. In this embodiment, the electrodes in each row share an interconnection or lead, and the electrodes in each column share an interconnection as well. By “interconnect” or “interconnection” or “lead” herein is meant a conductive path to and from the electrode, generally made of gold or copper, or other materials suitable for the formation of electrodes, as outlined above. Reference is made to FIG. 4.

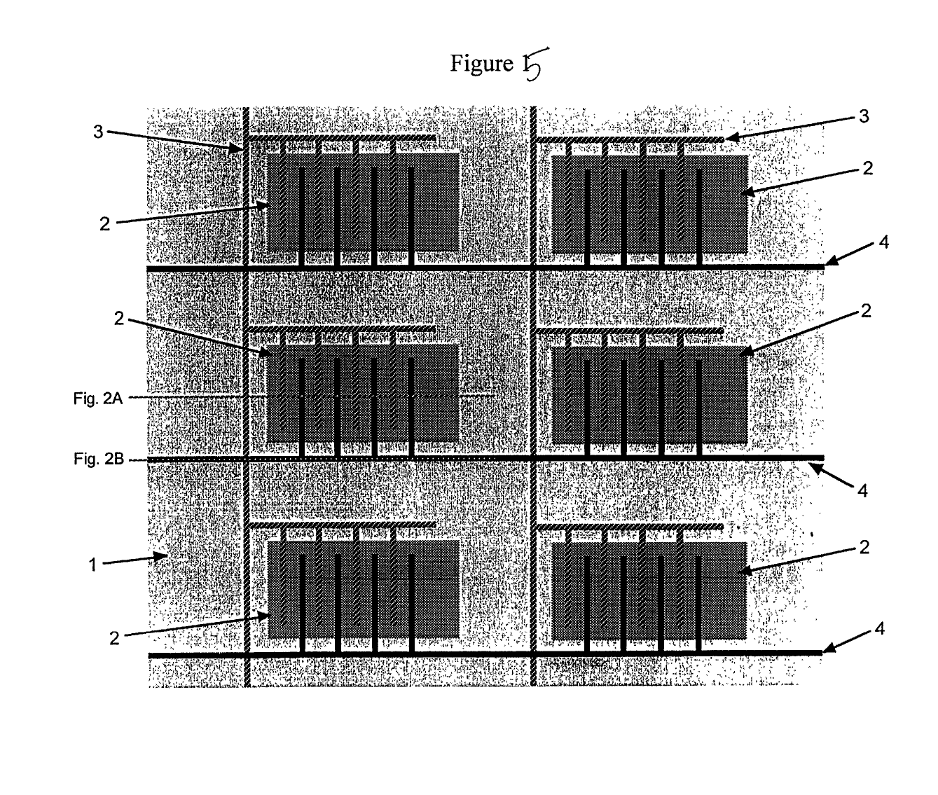

In a preferred embodiment, the two electrodes at each address are interdigitated, as is generally shown in FIG. 5, particularly when porous, polymeric pads are used, as described below. By embedding the input and output electrodes in the porous, polymeric pads (as shown in the Figures) the surface area of the input and output electrodes in contact with the porous, polymeric pad can be increased. Similarly, in embodiments in which the input and output electrodes protrude into the test site to contact the sample solution, the surface area of the input and output electrodes in contact with the sample solution is increased. This is advantageous in embodiments in which probe molecules are immobilized via linker moieties to the surface of the input and/or output electrodes. Furthermore, the surface area of the input and output electrodes can be increased by embedding a plurality of projections from a single input and/or output electrode into each porous, polymeric pad (or similarly in some embodiments, into the sample solution at each test site).

The plurality of projections from the input and output electrodes can also be interdigitated (as shown in the Figures). By varying the spacing and width of the interdigitated electrodes, the bioarrays of the present invention can be tuned to the specific detection scheme to be employed. For example, for electrical detection schemes (e.g, capacitance), small spacings between the input and output electrodes are desired. Preferably, the spacing between input and output electrodes is less than about 1 micron for the electrical detection devices of the present invention.

In this embodiment, the electrodes can comprise polymeric and optionally porous pads. The porous, polymeric pads of the apparatus of the invention are composed of materials including, but not limited to, polyacrylamide gel, agarose gel, polyethylene glycol, cellulose gel, sol gel, polypyrrole, carbon, carbides, oxides, nitrides, or other porous, polymeric materials known to those with skill in the art. In a preferred embodiment, the porous, polymeric pads comprise polyacrylamide gel.

Alternatively, in some embodiments, the electrodes comprise conjugated or conductive polymer or copolymer film, including, but not limited to, polypyrrole, polythiphene, polyaniline, polyfuran, polypyridine, polycarbazole, polyphenylene, poly(phenylenvinylene), polyfluorene, or polyindole, or their derivatives, their copolymers, and combinations thereof. In preferred embodiments, the material comprises a neutral pyrrole matrix. As more fully outlined below, thes conjugated polymers can be functionalized for the addition of capture binding ligands such as capture probes.

In a preferred embodiment, two dimensional arrays are provided with one electrode at each test site, and a counter or reference electrode can be shared by one or more of the electrodes. In this embodiment, the electrodes in each row share an interconnection or lead, and the electrodes in each column share an interconnection as well.

In a preferred embodiment, the devices of the invention comprise a plurality of microchannels. Thus, the devices of the invention include at least one microchannel or flow channel that allows the flow of sample from the sample inlet port to the other components or modules of the system. The collection of microchannels and wells is sometimes referred to in the art as a “mesoscale flow system”. As will be appreciated by those in the art, the flow channels may be configured in a wide variety of ways, depending on the use of the channel. For example, a single flow channel starting at the sample inlet port may be separated into a variety of smaller channels, such that the original sample is divided into discrete subsamples for parallel processing or analysis. Alternatively, several flow channels from different modules, for example the sample inlet port and a reagent storage module may feed together into a mixing chamber or a reaction chamber. As will be appreciated by those in the art, there are a large number of possible configurations; what is important is that the flow channels allow the movement of sample and reagents from one part of the device to another. For example, the path lengths of the flow channels may be altered as needed; for example, when mixing and timed reactions are required, longer and sometimes tortuous flow channels can be used.

In general, the microfluidic devices of the invention are generally referred to as “mesoscale” devices. The devices herein are typically designed on a scale suitable to analyze microvolumes, although in some embodiments large samples (e.g. cc's of sample) may be reduced in the device to a small volume for subsequent analysis. That is, “mesoscale” as used herein refers to chambers and microchannels that have cross-sectional dimensions on the order of 0.1 μm to 500 μm. The mesoscale flow channels and wells have preferred depths on the order of 0.1 μm to 100 μm, typically 2-50 μm. The channels have preferred widths on the order of 2.0 to 500 μm, more preferably 3-100 μm. For many applications, channels of 5-50 μm are useful. However, for many applications, larger dimensions on the scale of millimeters may be used. Similarly, chambers (sometimes also referred to herein as “wells”) in the substrates often will have larger dimensions, on the scale of a few millimeters.

In addition, it should be understood that while most of the discussion herein is directed to the use of planar substrates with microchannels and wells, other geometries can be used as well. For example, two or more planar substrates can be stacked to produce a three dimensional device, that can contain microchannels flowing within one plane or between planes; similarly, wells may span two or more substrates to allow for larger sample volumes. Thus for example, both sides of a substrate can be etched to contain microchannels; see for example U.S. Pat. Nos. 5,603,351 and 5,681,484, both of which are hereby incorporated by reference.

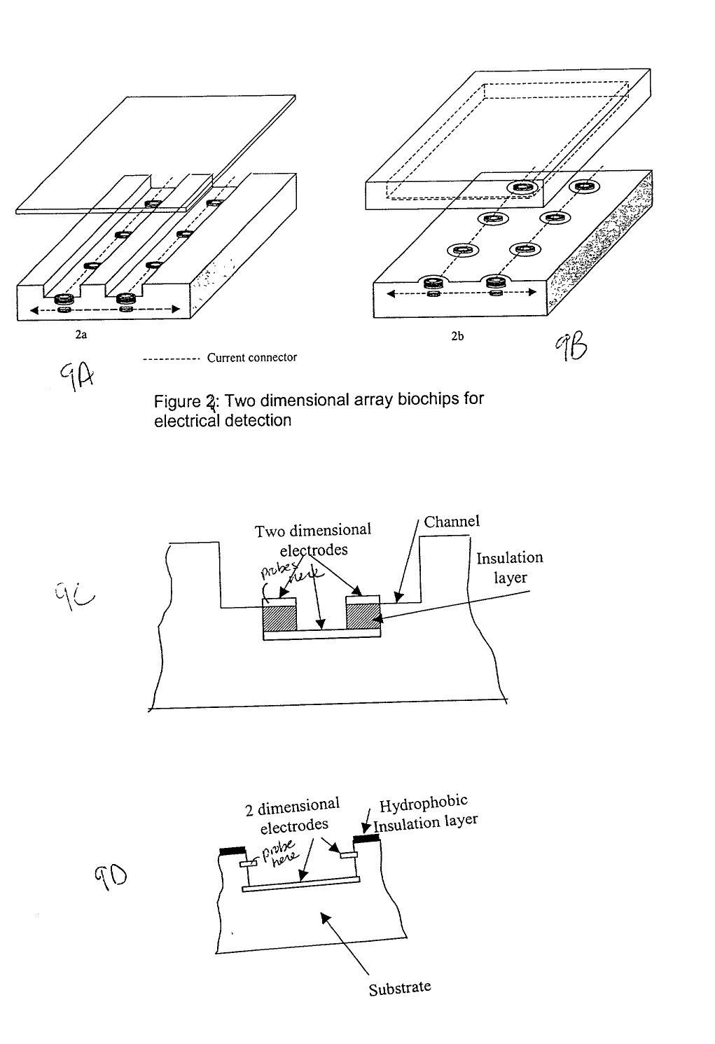

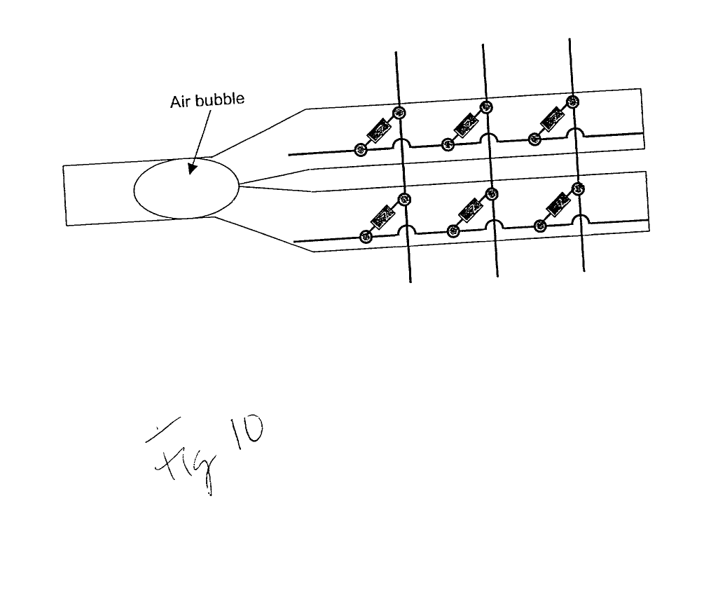

In a preferred embodiment, the microchannels encompass either a row or column of electrodes, as is generally depicted in FIGS. 9 and 10. The microchannels may each comprise a separate inlet port, or, as depicted in FIG. 10, share an inlet port that is then shut using a valve such as an air bubble. As will be appreciated by those in the art, other types of valves, known in the art, may be used as well.

In a preferred embodiment, the devices comprise two dimensional array of electrodes with microchannels, wherein the electrodes within a microchannel share a reference electrode, rather than having two electrodes at each test site.

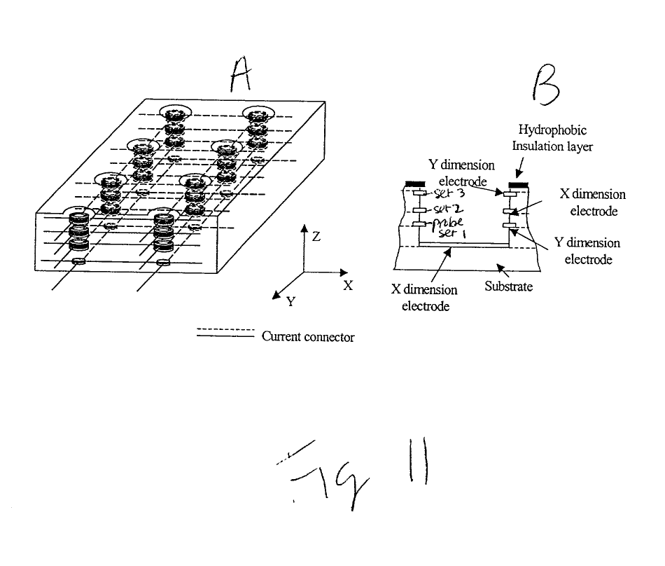

In a preferred embodiment, the devices of the invention comprise a three dimensional grid of electrodes. In this embodiment, the solid support comprises layers of two dimensional arrays. In this embodiment, the solid support comprises a number of layers, including a general solid support, layers of electrodes generally interspersed with insulating layers. In this embodiment, the matrix of electrodes are positioned within a matrix of wells, wherein each well comprises a plurality of electrodes in the Z dimension, as is generally depicted in FIGS. 11A and 11B.

In an alternative embodiment, an active driving circuit such as the one used in an active matrix liquid crystal display device can be built underneath or nearby each test well site to replace the electronic column and row drivers for x-y addressing such as the one used in the passive matrix liquid crystal display device.

The electrodes generally comprise capture binding ligands. By “capture binding ligand”, “binding ligand” or “binding species” herein is meant a compound that is used to probe for the presence of the target analyte, that will bind to the target analyte. There are two general ways that the assays of the invention are run. In a first embodiment, a capture binding ligand is used, and the target analyte is labeled; binding of the target analyte thus provides the label at the surface of the electrode. Alternatively, unlabeled target analytes are used, and a “sandwich” format is utilized; in this embodiment, there are at least two binding ligands used per target analyte molecule; a “capture” or “anchor” binding ligand (also referred to herein as a “capture probe”, particularly in reference to a nucleic acid binding ligand) that is attached to the detection electrode as described herein, and a soluble binding ligand (frequently referred to herein as a “signaling probe” or a “label probe”), that binds independently to the target analyte, and either directly or indirectly comprises at least one ETM.

Generally, the capture binding ligand allows the attachment of a target analyte to the detection electrode, for the purposes of detection. As is more fully outlined below, attachment of the target analyte to the capture binding ligand may be direct (i.e. the target analyte binds to the capture binding ligand) or indirect (one or more capture extender ligands may be used).

In a preferred embodiment, the binding is specific, and the binding ligand is part of a binding pair. By “specifically bind” herein is meant that the ligand binds the analyte, with specificity sufficient to differentiate between the analyte and other components or contaminants of the test sample. However, as will be appreciated by those in the art, it will be possible to detect analytes using binding that is not highly specific; for example, the systems may use different binding ligands, for example an array of different ligands, and detection of any particular analyte is via its “signature” of binding to a panel of binding ligands, similar to the manner in which “electronic noses” work. The binding should be sufficient to allow the analyte to remain bound under the conditions of the assay, including wash steps to remove non-specific binding. In some embodiments, for example in the detection of certain biomolecules, the binding constants of the analyte to the binding ligand will be at least about 10⁻⁴ to 10⁻⁶ M⁻¹, with at least about 10⁻⁵ to 10⁻⁹ being preferred and at least about 10⁻⁷ to 10 ⁻⁹ M⁻¹ being particularly preferred.

As will be appreciated by those in the art, the composition of the binding ligand will depend on the composition of the target analyte. Binding ligands to a wide variety of analytes are known or can be readily found using known techniques. For example, when the analyte is a single-stranded nucleic acid, the binding ligand is generally a substantially complementary nucleic acid. Alternatively, as is generally described in U.S. Pat. Nos. 5,270,163, 5,475,096, 5,567,588, 5,595,877, 5,637,459, 5,683,867, 5,705,337, and related patents, hereby incorporated by reference, nucleic acid “aptamers” can be developed for binding to virtually any target analyte. Similarly the analyte may be a nucleic acid binding protein and the capture binding ligand is either a single-stranded or double-stranded nucleic acid; alternatively, the binding ligand may be a nucleic acid binding protein when the analyte is a single or double-stranded nucleic acid. When the analyte is a protein, the binding ligands include proteins (particularly including antibodies or fragments thereof (FAbs, etc.)), small molecules, or aptamers, described above. Preferred binding ligand proteins include peptides. For example, when the analyte is an enzyme, suitable binding ligands include substrates, inhibitors, and other proteins that bind the enzyme, i.e. components of a multi-enzyme (or protein) complex. As will be appreciated by those in the art, any two molecules that will associate, preferably specifically, may be used, either as the analyte or the binding ligand. Suitable analyte/binding ligand pairs include, but are not limited to, antibodies/antigens, receptors/ligand, proteins/nucleic acids; nucleic acids/nucleic acids, enzymes/substrates and/or inhibitors, carbohydrates (including glycoproteins and glycolipids)/lectins, carbohydrates and other binding partners, proteins/proteins; and protein/small molecules. These may be wild-type or derivative sequences. In a preferred embodiment, the binding ligands are portions (particularly the extracellular portions) of cell surface receptors that are known to multimerize, such as the growth hormone receptor, glucose transporters (particularly GLUT4 receptor), transferrin receptor, epidermal growth factor receptor, low density lipoprotein receptor, high density lipoprotein receptor, leptin receptor, interleukin receptors including IL-1, IL-2, IL-3, IL-4, IL-5, IL-6, IL-7, IL-8, IL-9, IL-11, IL-12, IL-13, IL-15 and IL-17 receptors, VEGF receptor, PDGF receptor, EPO receptor, TPO receptor, ciliary neurotrophic factor receptor, prolactin receptor, and T-cell receptors. Similarly, there is a wide body of literature relating to the development of binding partners based on combinatorial chemistry methods.

In a preferred embodiment, the target analytes are nucleic acids and the capture binding ligands are nucleic acid probes (generally referred to herein as “capture probes”). Probes of the present invention are designed to be complementary to a target sequence (either the target sequence of the sample or to other probe sequences), such that hybridization of the target sequence and the probes of the present invention occurs. As outlined below, this complementarity need not be perfect; there may be any number of base pair mismatches which will interfere with hybridization between the target sequence and the single stranded nucleic acids of the present invention. However, if the number of mutations is so great that no hybridization can occur under even the least stringent of hybridization conditions, the sequence is not a complementary target sequence. Thus, by “substantially complementary” herein is meant that the probes are sufficiently complementary to the target sequences to hybridize under normal reaction conditions.

Generally, the nucleic acid compositions of the invention are useful as oligonucleotide probes. As is appreciated by those in the art, the length of the probe will vary with the length of the target sequence and the hybridization and wash conditions. Generally, oligonucleotide probes range from about 8 to about 50 nucleotides, with from about 10 to about 30 being preferred and from about 12 to about 25 being especially preferred. In some cases, very long probes may be used, e.g. 50 to 200-300 nucleotides in length.

A variety of hybridization conditions may be used in the present invention, including high, moderate and low stringency conditions; see for example Maniatis et al., Molecular Cloning: A Laboratory Manual, 2d Edition, 1989, and Short Protocols in Molecular Biology, ed. Ausubel, et al, hereby incorporated by reference. The hybridization conditions may also vary when a non-ionic backbone, i.e. PNA is used, as is known in the art. In addition, cross-linking agents may be added after target binding to cross-link, i.e. covalently attach, the two strands of the hybridization complex.

In this embodiment, when the binding ligand is a nucleic acid, preferred compositions and techniques are outlined in U.S. Pat. Nos. 5,591,578; 5,824,473; 5,705,348; 5,780,234 and 5,770,369; U.S. Ser. Nos. 08/873,598 08/911,589; WO 98/20162; WO98/12430; WO98/57158; WO 00/16089) WO99/57317; WO99/67425; WO00/24941; PCT US00/10903; WO00/38836; WO99/37819; WO99/57319 and PCTUS00/20476; and related materials, all of which are expressly incorporated by reference in their entirety.

The method of attachment of the capture binding ligands to the electrodes can be done in a variety of ways, depending on the composition of the capture binding ligand and the composition of the electrode. Both direct attachment (e.g. the capture binding ligand such as a nucleic acid probe is directly attached to the conductive polymer layer or gel pad layer), and indirect attachment, using an attachment linker, can be done. In general, both ways utilize functional groups on the capture binding ligands, the attachment linker, the gel polymer or the conductive polyer through the use of functional groups on each that can then be used for attachment. Preferred functional groups for attachment are amino groups, carboxy groups, oxo groups and thiol groups. These functional groups can then be attached, either directly or indirectly through the use of a linker, sometimes depicted herein as “Z”. Linkers are well known in the art; for example, homo-or hetero-bifunctional linkers as are well known (see 1994 Pierce Chemical Company catalog, technical section on cross-linkers, pages 155-200, incorporated herein by reference). Preferred modifications to the oligonucleotides useful in the practice of the invention include but are not limited to —OH, —NH₂, —SH, —COOR (where R=H, lower (C₁₋₁₂) alkyl, aryl, heterocyclic alkyl or aryl, or a metal ion), —CN, or —CHO. Immobilization of such derivatized probes is accomplished by direct attaching of the probe molecules on the electrode surface through a functional group such —OH, —SH, —NH₂.

Alternatively, probe molecules can be efficiently immobilized on the electrode surface through an intermediate species, termed a “spacer.” In these embodiments, the surface of the electrode 5 is first modified with an intermediate species that carries functional groups such as hydroxyl (—OH), amino (—NH₂), thiol (—SH), carboxyl ester (—COOR, where R=H, lower (C₁₋₁₂) alkyl, aryl, heterocyclic alkyl or aryl, or a metal ion), nitrile (—CN), or aldehylde (—CHO), which can react with the probe molecules functionalized with complementary members of the aforementioned anchoring groups.

In another embodiment, the surface of the electrodes 5 is covered with a layer of polymer matrix. In these embodiments, probe molecules are attached onto a supporting matrix on the surface of the electrodes using the functional chemistry mentioned above. The polymer matrix is preferably selected to be polypyrrole, polythiophene, polyaniline, polyacrylamide, agarose gel, polyethylene glycol, cellular, sol gels, dendrimers, metallic nanoparticles, carbon nanotubes, and their copolymers. To increase the probe loading capacity, porous matrix such as polyacrylamide, agarose, or sol gels are preferred.

In this way, capture binding ligands comprising proteins, lectins, nucleic acids, small organic molecules, carbohydrates, etc. can be added.

A preferred embodiment utilizes proteinaceous capture binding ligands. As is known in the art, any number of techniques may be used to attach a proteinaceous capture binding ligand to an attachment linker. A wide variety of techniques are known to add moieties to proteins.

A preferred embodiment utilizes nucleic acids as the capture binding ligand. While most of the following discussion focuses on nucleic acids, as will be appreciated by those in the art, many of the techniques outlined below apply in a similar manner to non-nucleic acid systems as well, and to systems that rely on attachment to surfaces other than metal electrodes.

The capture probe nucleic acid is covalently attached to the electrode, via an “attachment linker”, using a variety of techniques. By “covalently attached” herein is meant that two moieties are attached by at least one bond, including sigma bonds, pi bonds and coordination bonds. Preferred methods utilize conductive polymers or insulators as is generally described in WO 98/20162 and WO 99/57317, both of which are hereby expressly incorporated herein by reference in their entirety.

In a preferred embodiment, the electrodes comprise a self-assembled monolayer. As outlined herein, the efficiency of target analyte binding (for example, oligonucleotide hybridization) may increase when the analyte is at a distance from the electrode. Similarly, non-specific binding of biomolecules, including the target analytes, to an electrode is generally reduced when a monolayer is present. Thus, a monolayer facilitates the maintenance of the analyte away from the electrode surface. In addition, a monolayer serves to keep charged species away from the surface of the electrode. Thus, this layer helps to prevent electrical contact between the electrodes and the ETMs, or between the electrode and charged species within the solvent. Such contact can result in a direct “short circuit” or an indirect short circuit via charged species which may be present in the sample. Accordingly, the monolayer is preferably tightly packed in a uniform layer on the electrode surface, such that a minimum of “holes” exist. The monolayer thus serves as a physical barrier to block solvent accesibility to the electrode.

By “monolayer” or “self-assembled monolayer” or “SAM” herein is meant a relatively ordered assembly of molecules spontaneously chemisorbed on a surface, in which the molecules are oriented approximately parallel to each other and roughly perpendicular to the surface. A majority of the molecules includes a functional group that adheres to the surface, and a portion that interacts with neighboring molecules in the monolayer to form the relatively ordered array. A “mixed” monolayer comprises a heterogeneous monolayer, that is, where at least two different molecules make up the monolayer.

In general, the SAMs of the invention can be generated in a number of ways and comprise a number of different components, depending on the electrode surface and the system used. Preferred SAMs comprise insulating molecules, generally alkyl chains, such as are described in WO 99/57317, expressly incorporated herein by reference in its entirety.

In addition to the interconnects and electrodes, other electronic components can be added to the device as well, including, but not limited to, multplexers, filters, etc. One problem presented in conventional systems and methods is the difficulty in providing electrical connections (inputs and/or outputs) to a large number of electrodes, particularly if the electrodes form a dense or close packed array. Several solutions to this problem have been identified, and include the use of circuitry that allows signal processing either simultaneously as sets of parallel circuits and connections, line-sample array addressing, serially in a time-domain multiplexed manner, or in parallel or serially using frequency domain and/or time-domain based separation techniques, among other available techniques, as are outlined herein.

For example, a preferred method to connect a first multiplicity of circuits or lines on the chip to a smaller plurality of lines at a connector leading from the chip are to use a switching device such as a multiplexer (MUX) or relays to selectively couple circuits on the chip or board with circuits off the board.

The number of multiplexers will depend on the number of electrodes in the array. In one embodiment, a single MUX is utilized. In a preferred embodiment, a plurality of MUXs are used. This can be done in a variety of ways, as will be appreciated by those in the art; in one embodiment, “sectors” of electrodes are assigned to a particular MUX; thus for example, rows or columns of the array may each have their own MUX. Alternatively, submultiplexers are used; for example, a column or row is connected to a respective sub-multiplexer, with the sub-multiplexer outputs going to another submultiplexer.

In a preferred embodiment, the multiplexer includes a binary counter which receives the control signal via the connector pad. The control signal is preferably a pulsed signal such as a clock signal and generates a sequential count to drive the decoders.

In a preferred embodiment, another way to connect a multiplicity of electrodes on the substrate to a smaller number of connector pads leading “off chip” is to use row-column select signals to allow the selection of individual electrodes.

In a preferred embodiment, the array is divided into “sectors”, wherein a subset of the electrodes in the array have an associated MUX and preamplifier. Similarly, other components of the invention may be associated with sectors.

In a preferred embodiment, filters are used, including, but not limited to, time domain filters and frequency domain filters, and combinations.

In addition to the components outlined above, the devices of the invention can comprise any number of different components. As such, the invention provides microfluidic cassettes or devices that can be used to effect a number of manipulations on a sample to ultimately result in target analyte detection or quantification. These manipulations can include cell handling (cell concentration, cell lysis, cell removal, cell separation, etc.), separation of the desired target analyte from other sample components, chemical or enzymatic reactions on the target analyte, detection of the target analyte, etc. The devices of the invention can include one or more wells for sample manipulation, waste or reagents; microchannels to and between these wells, including microchannels containing electrophoretic separation matrices; valves to control fluid movement; on-chip pumps such as electroosmotic, electrohydrodynamic, or electrokinetic pumps; and detection systems comprising electrodes, as is more fully described below. The devices of the invention can be configured to manipulate one or multiple samples or analytes.

The devices of the invention can be made in a variety of ways, as will be appreciated by those in the art. See for example WO96/39260, directed to the formation of fluid-tight electrical conduits; U.S. Pat. No. 5,747,169, directed to sealing; EP 0637996 B1; EP 0637998 B1; WO96/39260; WO97/16835; WO98/13683; WO97/16561; WO97/43629; WO96/39252; WO96/15576; WO96/15450; WO97/37755; and WO97/27324; and U.S. Pat. Nos. 5,304,487; 5,071531; 5,061,336; 5,747,169; 5,296,375; 5,110,745; 5,587,128; 5,498,392; 5,643,738; 5,750,015; 5,726,026; 5,35,358; 5,126,022; 5,770,029; 5,631,337; 5,569,364; 5,135,627; 5,632,876; 5,593,838; 5,585,069; 5,637,469; 5,486,335; 5,755,942; 5,681,484; and 5,603,351, all of which are hereby incorporated by reference. Suitable fabrication techniques again will depend on the choice of substrate, but preferred methods include, but are not limited to, a variety of micromachining and microfabrication techniques, including film deposition processes such as spin coating, chemical vapor deposition, laser fabrication, photolithographic and other etching techniques using either wet chemical processes or plasma processes, embossing, injection molding and bonding techniques (see U.S. Pat. No. 5,747,169, hereby incorporated by reference). In addition, there are printing techniques for the creation of desired fluid guiding pathways; that is, patterns of printed material can permit directional fluid transport. Thus, the build-up of “ink” can serve to define a flow channel. In addition, the use of different “inks” or “pastes” can allow different portions of the pathways having different flow properties. For example, materials can be used to change solute/solvent RF values (the ratio of the distance moved by a particular solute to that moved by a solvent front). For example, printed fluid guiding pathways can be manufactured with a printed layer or layers comprised of two different materials, providing different rates of fluid transport. Multi-material fluid guiding pathways can be used when it is desirable to modify retention times of reagents in fluid guiding pathways. Furthermore, printed fluid guiding pathways can also provide regions containing reagent substances, by including the reagents in the “inks” or by a subsequent printing step. See for example U.S. Pat. No. 5,795,453, herein incorporated by reference in its entirety.

In a preferred embodiment, the solid substrate is configured for handling a single sample that may contain a plurality of target analytes. That is, a single sample is added to the device and the sample may either be aliquoted for parallel processing for detection of the analytes or the sample may be processed serially, with individual targets being detected in a serial fashion. In addition, samples may be removed periodically or from different locations for in line sampling.

In a preferred embodiment, the solid substrate is configured for handling multiple samples, each of which may contain one or more target analytes. In general, in this embodiment, each sample is handled individually; that is, the manipulations and analyses are done in parallel, with preferably no contact or contamination between them. Alternatively, there may be some steps in common; for example, it may be desirable to process different samples separately but detect all of the target analytes on a single detection electrode, as described below.

Once made, the devices of the invention find use in a variety of applications. In particular, the compositions of the invention find use in hybridization assays. As will be appreciated by those in the art, electrodes can be made that have a single species of nucleic acid, i.e. a single nucleic acid sequence, or multiple nucleic acid species.

Recent focus has been on the analysis of the relationship between genetic variation and phenotype by making use of polymorphic DNA markers. Previous work utilized short tandem repeats (STRs) as polymorphic positional markers; however, recent focus is on the use of single nucleotide polymorphisms (SNPs), which occur at an average frequency of more than 1 per kilobase in human genomic DNA. Some SNPs, particularly those in and around coding sequences, are likely to be the direct cause of therapeutically relevant phenotypic variants and/or disease predisposition. There are a number of well known polymorphisms that cause clinically important phenotypes; for example, the apoE2/3/4 variants are associated with different relative risk of Alzheimer's and other diseases (see Cordor et al., Science 261(1993). Multiplex PCR amplification of SNP loci with subsequent hybridization to oligonucleotide arrays has been shown to be an accurate and reliable method of simultaneously genotyping at least hundreds of SNPs

The present invention is directed to methods of determining the sequence of a target nucleic acid at a particular position, using electrochemical detection on an electrode. The invention preferably includes the detection (and optionally quantification) of differences or variations of sequences (e.g. SNPs) using electrode arrays for detection of the variation.

As is known in the art, there are a number of techniques that can be used to detect or determine the identity of a base at a particular location in a target nucleic acid, including, but not limited to, the use of temperature, competitive hybridization of perfect and imperfect probes to the target sequence, sequencing by synthesis, for example using single base extension techniques (sometimes referred to as “minisequencing”), the oligonucleotide ligase amplification (OLA) reaction, rolling circle amplification (RCA), allelic PCR, competitive hybridization and Invader™ technologies. In addition, the present invention is directed to a novel invention that capitalizes on novel properties of surface-bound arrays, and uses “competimers” to reduce non-specific binding.

Thus, the compositions of the present invention may be used in a variety of research, clinical, quality control, or field testing settings.

In a preferred embodiment, the probes are used in genetic diagnosis. For example, probes can be made using the techniques disclosed herein to detect target sequences such as the gene for nonpolyposis colon cancer, the BRCA1 breast cancer gene, P53, which is a gene associated with a variety of cancers, the Apo E4 gene that indicates a greater risk of Alzheimer's disease, allowing for easy presymptomatic screening of patients, mutations in the cystic fibrosis gene, or any of the others well known in the art.

In an additional embodiment, viral and bacterial detection is done using the complexes of the invention. In this embodiment, probes are designed to detect target sequences from a variety of bacteria and viruses. For example, current blood-screening techniques rely on the detection of anti-HIV antibodies. The methods disclosed herein allow for direct screening of clinical samples to detect HIV nucleic acid sequences, particularly highly conserved HIV sequences. In addition, this allows direct monitoring of circulating virus within a patient as an improved method of assessing the efficacy of anti-viral therapies. Similarly, viruses associated with leukemia, HTLV-I and HTLV-II, may be detected in this way. Bacterial infections such as tuberculosis, clymidia and other sexually transmitted diseases, may also be detected, for example using ribosomal RNA (rRNA) as the target sequences.

In a preferred embodiment, the nucleic acids of the invention find use as probes for toxic bacteria in the screening of water and food samples. For example, samples may be treated to lyse the bacteria to release its nucleic acid (particularly rRNA), and then probes designed to recognize bacterial strains, including, but not limited to, such pathogenic strains as, Salmonella, Campylobacter, Vibrio cholerae, Leishmania, enterotoxic strains of E. coli, and Legionnaire's disease bacteria. Similarly, bioremediation strategies may be evaluated using the compositions of the invention.

In a further embodiment, the probes are used for forensic “DNA fingerprinting” to match crime-scene DNA against samples taken from victims and suspects.

In an additional embodiment, the probes in an array are used for sequencing by hybridization.

Thus, the present invention provides for extremely specific and sensitive probes, which may, in some embodiments, detect target sequences without removal of unhybridized probe. This will be useful in the generation of automated gene probe assays.

Alternatively, the compositions of the invention are useful to detect successful gene amplification in PCR, thus allowing successful PCR reactions to be an indication of the presence or absence of a target sequence. PCR may be used in this manner in several ways. For example, in one embodiment, the PCR reaction is done as is known in the art, and then added to a composition of the invention comprising the target nucleic acid with a ETM, covalently attached to an electrode via a conductive oligomer with subsequent detection of the target sequence. Alternatively, PCR is done using nucleotides labelled with a ETM, either in the presence of, or with subsequent addition to, an electrode with a conductive oligomer and a target nucleic acid. Binding of the PCR product containing ETMs to the electrode composition will allow detection via electron transfer. Finally, the nucleic acid attached to the electrode via a conductive polymer may be one PCR primer, with addition of a second primer labelled with an ETM. Elongation results in double stranded nucleic acid with a ETM and electrode covalently attached. In this way, the present invention is used for PCR detection of target sequences.

In a preferred embodiment, the arrays are used for mRNA detection. A preferred embodiment utilizes either capture probes or capture extender probes that hybridize close to the 3′ polyadenylation tail of the mRNAs. This allows the use of one species of target binding probe for detection, i.e. the probe contains a poly-T portion that will bind to the poly-A tail of the mRNA target. Generally, the probe will contain a second portion, preferably non-poly-T, that will bind to the detection probe (or other probe). This allows one target-binding probe to be made, and thus decreases the amount of different probe synthesis that is done.

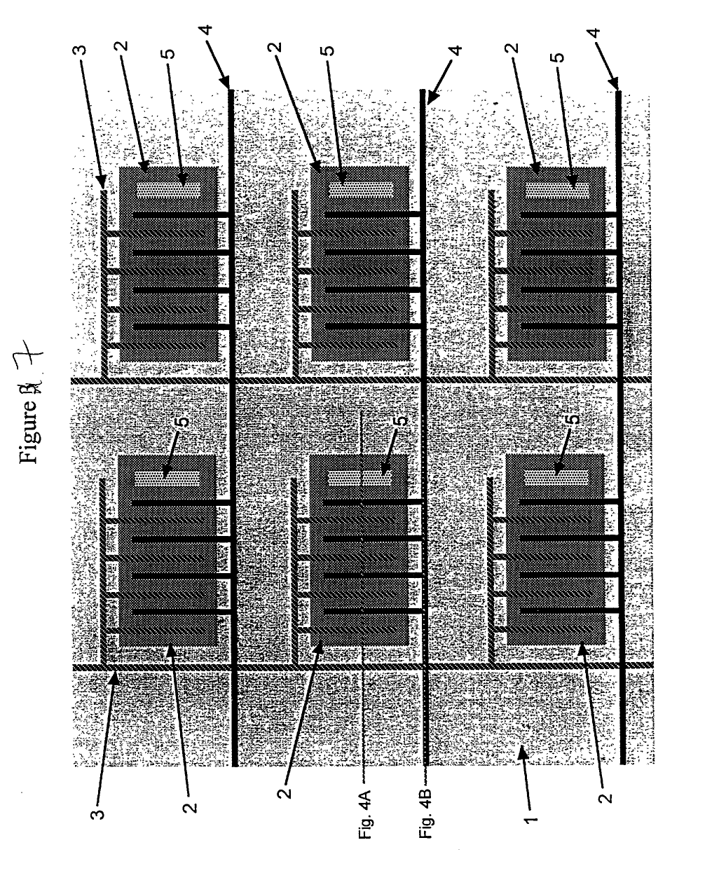

In a preferred embodiment, the use of restriction enzymes and ligation methods allows the creation of “universal” arrays. In this embodiment, monolayers comprising capture probes that comprise restriction endonuclease ends, as is generally depicted in FIG. 7 of PCT US97/20014. By utilizing complementary portions of nucleic acid, while leaving “sticky ends”, an array comprising any number of restriction endonuclease sites is made. Treating a target sample with one or more of these restriction endonucleases allows the targets to bind to the array. This can be done without knowing the sequence of the target. The target sequences can be ligated, as desired, using standard methods such as ligases, and the target sequence detected, using either standard labels or the methods of the invention.

As will be appreciated by those in the art, there are a variety of electronic and electrochemical detection techniques that can be used. In some embodiments, (e.g. electrochemical detection), hybridization complexes are formed that comprise a target sequence and a capture probe. The target sequence can comprise an electrochemically active reporter (also referred to herein as an electron transfer moiety (ETM)), such as a transition metal complex, defined below. Alternatively, in “sandwich” formats, the hybridization complex further comprises a label probe, that hybridizes to a domain of the target sequence, and comprises the label.

The terms “electron donor moiety”, “electron acceptor moiety”, and “ETMs” (ETMs) or grammatical equivalents herein refers to molecules capable of electron transfer under certain conditions. It is to be understood that electron donor and acceptor capabilities are relative; that is, a molecule which can lose an electron under certain experimental conditions will be able to accept an electron under different experimental conditions. It is to be understood that the number of possible electron donor moieties and electron acceptor moieties is very large, and that one skilled in the art of electron transfer compounds will be able to utilize a number of compounds in the present invention. Preferred ETMs include, but are not limited to, transition metal complexes, organic ETMs, and electrodes.

In a preferred embodiment, the ETMs are transition metal complexes. Transition metals are those whose atoms have a partial or complete d shell of electrons. Suitable transition metals for use in the invention include, but are not limited to, cadmium (Cd), copper (Cu), cobalt (Co), palladium (Pd), zinc (Zn), iron (Fe), ruthenium (Ru), rhodium (Rh), osmium (Os), rhenium (Re), platinium (Pt), scandium (Sc), titanium (Ti), Vanadium (V), chromium (Cr), manganese (Mn), nickel (Ni), Molybdenum (Mo), technetium (Tc), tungsten (W), and iridium (Ir). That is, the first series of transition metals, the platinum metals (Ru, Rh, Pd, Os, Ir and Pt), along with Fe, Re, W, Mo and Tc, are preferred. Particularly preferred are ruthenium, rhenium, osmium, platinium, cobalt and iron.

The transition metals are complexed with a variety of ligands, L, to form suitable transition metal complexes, as is well known in the art. L are the co-ligands, that provide the coordination atoms for the binding of the metal ion. As will be appreciated by those in the art, the number and nature of the co-ligands will depend on the coordination number of the metal ion. Mono-, di- or polydentate co-ligands may be used at any position. Thus, for example, when the metal has a coordination number of six, the L from the terminus of the conductive oligomer, the L contributed from the nucleic acid, and r, add up to six. Thus, when the metal has a coordination number of six, r may range from zero (when all coordination atoms are provided by the other two ligands) to four, when all the co-ligands are monodentate. Thus generally, r will be from 0 to 8, depending on the coordination number of the metal ion and the choice of the other ligands.

In one embodiment, the metal ion has a coordination number of six and both the ligand attached to the conductive oligomer and the ligand attached to the nucleic acid are at least bidentate; that is, r is preferably zero, one (i.e. the remaining co-ligand is bidentate) or two (two monodentate co-ligands are used).

As will be appreciated in the art, the co-ligands can be the same or different. Suitable ligands fall into two categories: ligands which use nitrogen, oxygen, sulfur, carbon or phosphorus atoms (depending on the metal ion) as the coordination atoms (generally referred to in the literature as sigma (σ) donors) and organometallic ligands such as metallocene ligands (generally referred to in the literature as pi (π) donors, and depicted herein as Lₘ). Suitable nitrogen donating ligands are well known in the art and include, but are not limited to, NH₂; NHR; NRR′; pyridine; pyrazine; isonicotinamide; imidazole; bipyridine and substituted derivatives of bipyridine; terpyridine and substituted derivatives; phenanthrolines, particularly 1,10-phenanthroline (abbreviated phen) and substituted derivatives of phenanthrolines such as 4,7-dimethylphenanthroline and dipyridol[3,2-a:2′,3′-c]phenazine (abbreviated dppz); dipyridophenazine; 1,4,5,8,9,12-hexaazatriphenylene (abbreviated hat); 9,10-phenanthrenequinone diimine (abbreviated phi); 1,4,5,8-tetraazaphenanthrene (abbreviated tap); 1,4,8,11-tetra-azacyclotetradecane (abbreviated cyclam), EDTA, EGTA and isocyanide. Substituted derivatives, including fused derivatives, may also be used. In some embodiments, porphyrins and substituted derivatives of the porphyrin family may be used. See for example, Comprehensive Coordination Chemistry, Ed. Wilkinson et al., Pergammon Press, 1987, Chapters 13.2 (pp73-98), 21.1 (pp. 813-898) and 21.3 (pp 915-957), all of which are hereby expressly incorporated by reference.

Suitable sigma donating ligands using carbon, oxygen, sulfur and phosphorus are known in the art. For example, suitable sigma carbon donors are found in Cotton and Wilkenson, Advanced Organic Chemistry, 5th Edition, John Wiley & Sons, 1988, hereby incorporated by reference; see page 38, for example. Similarly, suitable oxygen ligands include crown ethers, water and others known in the art. Phosphines and substituted phosphines are also suitable; see page 38 of Cotton and Wilkenson.

The oxygen, sulfur, phosphorus and nitrogen-donating ligands are attached in such a manner as to allow the heteroatoms to serve as coordination atoms.

In a preferred embodiment, organometallic ligands are used. In addition to purely organic compounds for use as redox moieties, and various transition metal coordination complexes with δ-bonded organic ligand with donor atoms as heterocyclic or exocyclic substituents, there is available a wide variety of transition metal organometallic compounds with π-bonded organic ligands (see Advanced Inorganic Chemistry, 5th Ed., Cotton & Wilkinson, John Wiley & Sons, 1988, chapter 26; Organometallics, A Concise Introduction, Elschenbroich et al., 2nd Ed., 1992, VCH; and Comprehensive Organometallic Chemistry II, A Review of the Literature 1982-1994, Abel et al. Ed., Vol. 7, chapters 7, 8, 10 & 11, Pergamon Press, hereby expressly incorporated by reference). Such organometallic ligands include cyclic aromatic compounds such as the cyclopentadienide ion [C₅H₅(−1)] and various ring substituted and ring fused derivatives, such as the indenylide (−1) ion, that yield a class of bis(cyclopentadieyl) metal compounds, (i.e. the metallocenes); see for example Robins et al., J. Am. Chem. Soc. 104:1882-1893 (1982); and Gassman et al., J. Am. Chem. Soc. 108:4228-4229 (1986), incorporated by reference. Of these, ferrocene [(C₅H₅)₂Fe] and its derivatives are prototypical examples which have been used in a wide variety of chemical (Connelly et al., Chem. Rev. 96:877-910 (1996), incorporated by reference) and electrochemical (Geiger et al., Advances in Organometallic Chemistry 23:1-93; and Geiger et al., Advances in Organometallic Chemistry 24:87, incorporated by reference) electron transfer or “redox” reactions. Metallocene derivatives of a variety of the first, second and third row transition metals are potential candidates as redox moieties that are covalently attached to either the ribose ring or the nucleoside base of nucleic acid. Other potentially suitable organometallic ligands include cyclic arenes such as benzene, to yield bis(arene)metal compounds and their ring substituted and ring fused derivatives, of which bis(benzene)chromium is a prototypical example, Other acyclic π-bonded ligands such as the allyl(−1) ion, or butadiene yield potentially suitable organometallic compounds, and all such ligands, in conjuction with other π-bonded and δ-bonded ligands constitute the general class of organometallic compounds in which there is a metal to carbon bond. Electrochemical studies of various dimers and oligomers of such compounds with bridging organic ligands, and additional non-bridging ligands, as well as with and without metal-metal bonds are potential candidate redox moieties in nucleic acid analysis.

When one or more of the co-ligands is an organometallic ligand, the ligand is generally attached via one of the carbon atoms of the organometallic ligand, although attachment may be via other atoms for heterocyclic ligands. Preferred organometallic ligands include metallocene ligands, including substituted derivatives and the metalloceneophanes (see page 1174 of Cotton and Wilkenson, supra). For example, derivatives of metallocene ligands such as methylcyclopentadienyl, with multiple methyl groups being preferred, such as pentamethylcyclopentadienyl, can be used to increase the stability of the metallocene. In a preferred embodiment, only one of the two metallocene ligands of a metallocene are derivatized.

As described herein, any combination of ligands may be used. Preferred combinations include: a) all ligands are nitrogen donating ligands; b) all ligands are organometallic ligands; and c) the ligand at the terminus of the conductive oligomer is a metallocene ligand and the ligand provided by the nucleic acid is a nitrogen donating ligand, with the other ligands, if needed, are either nitrogen donating ligands or metallocene ligands, or a mixture.

In addition to transition metal complexes, other organic electron donors and acceptors may be covalently attached to the nucleic acid for use in the invention. These organic molecules include, but are not limited to, riboflavin, xanthene dyes, azine dyes, acridine orange, N,N′-dimethyl-2,7-diazapyrenium dichloride (DAP²⁺), methylviologen, ethidium bromide, quinones such as N,N′-dimethylanthra(2,1,9-def:6,5,10-d′e′f′)diisoquinoline dichloride (ADIQ²⁺); porphyrins ([meso-tetrakis(N-methyl-x-pyridinium)porphyrin tetrachloride], varlamine blue B hydrochloride, Bindschedler's green; 2,6-dichloroindophenol, 2,6-dibromophenolindophenol; Brilliant crest blue (3-amino-9-dimethyl-amino-10-methylphenoxyazine chloride), methylene blue; Nile blue A (aminoaphthodiethylaminophenoxazine sulfate), indigo-5,5′,7,7′-tetrasulfonic acid, indigo-5,5′,7-trisulfonic acid; phenosafranine, indigo-5-monosulfonic acid; safranine T; bis(dimethylglyoximato)-iron(II) chloride; induline scarlet, neutral red, anthracene, coronene, pyrene, 9-phenylanthracene, rubrene, binaphthyl, DPA, phenothiazene, fluoranthene, phenanthrene, chrysene, 1,8-diphenyl-1,3,5,7-octatetracene, naphthalene, acenaphthalene, perylene, TMPD and analogs and subsitituted derivatives of these compounds.

In one embodiment, the electron donors and acceptors are redox proteins as are known in the art. However, redox proteins in many embodiments are not preferred.

The choice of the specific ETMs will be influenced by the type of electron transfer detection used, as is generally outlined below. Preferred ETMs are metallocenes, with ferrocene being particularly preferred.

In a preferred embodiment, a plurality of ETMs are used.

The ETMs are attached to nucleic acids, target analytes, or soluble binding ligands as is generally outlined in WO 98/20162, hereby expressly incorporated by reference in its entirety.

Alternatively, reporterless or labelless systems are used. In this embodiment, two electrodes are used to measure changes in capacitance or impedance as a result of target analyte binding. See generally U.S. Ser. Nos. 09/458,533, filed Dec. 9, 1999 and CPT US00/33497, both of which are expressly incorporated by reference.

The reactions outlined herein may be accomplished in a variety of ways, as will be appreciated by those in the art. Components of the reaction may be added simultaneously, or sequentially, in any order, with preferred embodiments outlined below. In addition, the reaction may include a variety of other reagents may be included in the assays. These include reagents like salts, buffers, neutral proteins, e.g. albumin, detergents, etc which may be used to facilitate optimal hybridization and detection, and/or reduce non-specific or background interactions. Also reagents that otherwise improve the efficiency of the assay, such as protease inhibitors, nuclease inhibitors, anti-microbial agents, etc., may be used, depending on the sample preparation methods and purity of the target.

As will be appreciated by those in the art, electrolyte solutions can be used. In the practice of the invention, a high-density, x-y addressable probe array is exposed to an electrolyte solution containing a target molecule for a time and under conditions sufficient for the target to bind to a probe present in at least one of the particular addresses of the column-and row addressable array. A voltage potential or other electric signal is applied to the each of the electrodes comprising each of the addressable sites through the x-y addressable column and row electrodes. Changes in the electrical properties or electrical signals from a particular electrode at a particular site in the x-y addressable array arising from interactions between probe molecules on the electrode and target molecules in the solution are detected to determine the presence and concentration of the target molecules in the solution.

Electrolyte solutions useful in the apparatus and methods of the invention include any electrolyte solution at physiologically-relevant ionic strength (equivalent to about 0.15M NaCl) and neutral pH. Non-limiting examples of electrolyte solutions useful with the apparatus and methods of the invention include but are not limited to phosphate buffered saline, HEPES buffered solutions, and sodium bicarbonate buffered solutions. In alternative embodiments useful for electrical detection methods provided by the invention, the electrolyte solution comprises metal cations or polymerized cations that are ion conductive and capable of reacting with probes or probe-target complexes.

Detection of electron transfer is generally initiated electronically, with voltage being preferred. A potential is applied to the assay complex. Precise control and variations in the applied potential can be via a potentiostat and either a three electrode system (one reference, one sample (or working) and one counter electrode) or a two electrode system (one sample and one counter electrode). This allows matching of applied potential to peak potential of the system which depends in part on the choice of ETMs (when reporters are used) and in part on the other system components, the composition and integrity of the monolayer, and what type of reference electrode is used. As described herein, ferrocene is a preferred ETM

In some embodiments, co-reductants or co-oxidants are used as is generally described in WO00/16089, hereby expressly incorporated by reference.

A variety of detection methods may be used, including, but not limited to, optical detection (as a result of spectral changes upon changes in redox states), which includes fluorescence, phosphorescence, luminescence, chemiluminescence, electrochemiluminescence, and refractive index; and electronic detection, including, but not limited to, amperommetry, voltammetry, capacitance and impedence. These methods include time or frequency dependent methods based on AC or DC currents, pulsed methods, lock-in techniques, filtering (high pass, low pass, band pass), and time-resolved techniques including time-resolved fluorescence.

In one embodiment, the efficient transfer of electrons from the ETM to the electrode results in stereotyped changes in the redox state of the ETM. With many ETMs including the complexes of ruthenium containing bipyridine, pyridine and imidazole rings, these changes in redox state are associated with changes in spectral properties. Significant differences in absorbance are observed between reduced and oxidized states for these molecules. See for example Fabbrizzi et al., Chem. Soc. Rev. 1995 pp197-202). These differences can be monitored using a spectrophotometer or simple photomultiplier tube device.

In this embodiment, possible electron donors and acceptors include all the derivatives listed above for photoactivation or initiation. Preferred electron donors and acceptors have characteristically large spectral changes upon oxidation and reduction resulting in highly sensitive monitoring of electron transfer. Such examples include Ru(NH₃)₄py and Ru(bpy)₂im as preferred examples. It should be understood that only the donor or acceptor that is being monitored by absorbance need have ideal spectral characteristics.

In a preferred embodiment, the electron transfer is detected fluorometrically. Numerous transition metal complexes, including those of ruthenium, have distinct fluorescence properties. Therefore, the change in redox state of the electron donors and electron acceptors attached to the nucleic acid can be monitored very sensitively using fluorescence, for example with Ru(4,7-biphenyl₂-phenanthroline)₃²⁺. The production of this compound can be easily measured using standard fluorescence assay techniques. For example, laser induced fluorescence can be recorded in a standard single cell fluorimeter, a flow through “on-line” fluorimeter (such as those attached to a chromatography system) or a multi-sample “plate-reader” similar to those marketed for 96-well immuno assays.

Alternatively, fluorescence can be measured using fiber optic sensors with nucleic acid probes in solution or attached to the fiber optic. Fluorescence is monitored using a photomultiplier tube or other light detection instrument attached to the fiber optic. The advantage of this system is the extremely small volumes of sample that can be assayed.

In addition, scanning fluorescence detectors such as the Fluorlmager sold by Molecular Dynamics are ideally suited to monitoring the fluorescence of modified nucleic acid molecules arrayed on solid surfaces. The advantage of this system is the large number of electron transfer probes that can be scanned at once using chips covered with thousands of distinct nucleic acid probes.

Many transition metal complexes display fluorescence with large Stokes shifts. Suitable examples include bis- and trisphenanthroline complexes and bis- and trisbipyridyl complexes of transition metals such as ruthenium (see Juris, A., Balzani, V., et. al. Coord. Chem. Rev., V. 84, p. 85-277, 1988). Preferred examples display efficient fluorescence (reasonably high quantum yields) as well as low reorganization energies. These include Ru(4,7-biphenyl₂-phenanthroline)₃²⁺, Ru(4,4′-diphenyl-2,2′-bipyridine)₃²⁺and platinum complexes (see Cummings et al., J. Am. Chem. Soc. 118:1949-1960 (1996), incorporated by reference). Alternatively, a reduction in fluorescence associated with hybridization can be measured using these systems.

In a further embodiment, electrochemiluminescence is used as the basis of the electron transfer detection. With some ETMs such as Ru²⁺(bpy)₃, direct luminescence accompanies excited state decay. Changes in this property are associated with nucleic acid hybridization and can be monitored with a simple photomultiplier tube arrangement (see Blackburn, G. F. Clin. Chem. 37: 1534-1539 (1991); and Juris et al., supra.

In a preferred embodiment, electronic detection is used, including amperommetry, voltammetry, capacitance, and impedence. Suitable techniques include, but are not limited to, electrogravimetry; coulometry (including controlled potential coulometry and constant current coulometry); voltametry (cyclic voltametry, pulse voltametry (normal pulse voltametry, square wave voltametry, differential pulse voltametry, Osteryoung square wave voltametry, and coulostatic pulse techniques); stripping analysis (aniodic stripping analysis, cathiodic stripping analysis, square wave stripping voltammetry); conductance measurements (electrolytic conductance, direct analysis); time-dependent electrochemical analyses (chronoamperometry, chronopotentiometry, cyclic chronopotentiometry and amperometry, AC polography, chronogalvametry, and chronocoulometry); AC impedance measurement; capacitance measurement; AC voltametry; and photoelectrochemistry.

In a preferred embodiment, monitoring electron transfer is via amperometric detection. This method of detection involves applying a potential (as compared to a separate reference electrode) between the nucleic acid-conjugated electrode and a reference (counter) electrode in the sample containing target genes of interest. Electron transfer of differing efficiencies is induced in samples in the presence or absence of target nucleic acid; that is, the presence or absence of the target nucleic acid, and thus the label probe, can result in different currents.

The device for measuring electron transfer amperometrically involves sensitive current detection and includes a means of controlling the voltage potential, usually a potentiostat. This voltage is optimized with reference to the potential of the electron donating complex on the label probe. Possible electron donating complexes include those previously mentioned with complexes of iron, osmium, platinum, cobalt, rhenium and ruthenium being preferred and complexes of iron being most preferred.

In a preferred embodiment, alternative electron detection modes are utilized. For example, potentiometric (or voltammetric) measurements involve non-faradaic (no net current flow) processes and are utilized traditionally in pH and other ion detectors. Similar sensors are used to monitor electron transfer between the ETM and the electrode. In addition, other properties of insulators (such as resistance) and of conductors (such as conductivity, impedance and capicitance) could be used to monitor electron transfer between ETM and the electrode. Finally, any system that generates a current (such as electron transfer) also generates a small magnetic field, which may be monitored in some embodiments.

It should be understood that one benefit of the fast rates of electron transfer observed in the compositions of the invention is that time resolution can greatly enhance the signal-to-noise results of monitors based on absorbance, fluorescence and electronic current. The fast rates of electron transfer of the present invention result both in high signals and stereotyped delays between electron transfer initiation and completion. By amplifying signals of particular delays, such as through the use of pulsed initiation of electron transfer and “lock-in” amplifiers of detection, and Fourier transforms.

In a preferred embodiment, electron transfer is initiated using alternating current (AC) methods. Without being bound by theory, it appears that ETMs, bound to an electrode, generally respond similarly to an AC voltage across a circuit containing resistors and capacitors.

When reporters such as ETMs are not used, other initiation/detection systems may be preferred. In this embodiment of the present invention, molecular interactions between immobilized probe molecules and target molecules in a sample mixture are detected by detecting an electrical signal using AC impedance. In other embodiments, such molecular interactions are detected by detecting an electrical signal using an electrical or electrochemical detection method selected from the group consisting of impedance spectroscopy, cyclic voltammetry, AC voltammetry, pulse voltammetry, square wave voltammetry, AC voltammetry, hydrodynamic modulation voltammetry, conductance, potential step method, potentiometric measurements, amperometric measurements, current step method, other steady-state or transient measurement methods, and combinations thereof.

In one embodiment of the apparatus of the present invention, the means for producing electrical impedance at each test electrode is accomplished using a Model 1260 Impedance/Gain Phase Analyzer with Model 1287 Electrochemical Interface (Solartron Inc., Houston, Tex.). Other electrical impedance measurement means include, but are not limited to, transient methods using AC signal perturbation superimposed upon a DC potential applied to an electrochemical cell such as AC bridge and AC voltammetry. The measurements can be conducted at any particular frequency that specifically produces electrical signal changes that are readily detected or otherwise determined to be advantageous. Such particular frequencies are advantageously determined by scanning frequencies to ascertain the frequency producing, for example, the largest difference in electrical signal. The means for detecting changes in impedance at each test site electrode as a result of molecular interactions between probe and target molecules can be accomplished by using any of the above-described instruments.

Several preferred embodiments are shown below with reference to the figures.

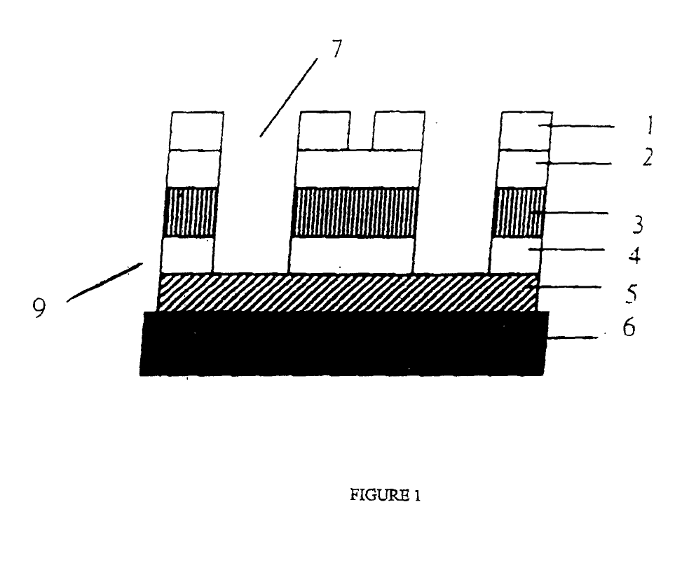

FIG. 1 illustrates a schematic representation of a cross-section view of the device platform 9 of the present invention. The device 9 is built on a solid supporting substrate 6, as outlined above, preferably of a non-porous substrate. Patterned conductive electrodes 5 are fabricated on top of the solid supporting substrate.

The devices are advantageously formed by standard fabrication techniques used in semiconductor manufacturing. Non-limiting examples of methods for producing solid substrates comprising the device platforms of the invention include but are not limited to thermal evaporation, wire bonding, metallization (evaporation, plating, sputtering over a shadow mask), dielectric deposition (by plasma, chemical vapor deposition or sputtering), wet or dry chemical etching, reactive ion etching, or liftoff after the desired pattern has been defined using conventional photolithography.

A layer of insulative dielectric material 4 as described above is placed on top of the patterned conductive electrodes 5. An optional layer of conductive metal 3 is placed over the insulative dielectric material 4. This layer constitutes a reference electrode. In a preferred embodiment, the conductive metal layer 3 is silver, which is then advantageously converted to silver/silver chloride at a later stage in manufacturing. A second layer of insulative dielectric material 2 is then placed on top of the conductive electrode layer 3. In embodiments not comprising a reference electrode 3, a continuous dielectric layer 2 comprising layers 2 and 4 as set forth herein are deposited. The second layer of insulative dielectric material 2 is optionally made of the same materials as the insulative layer 4.

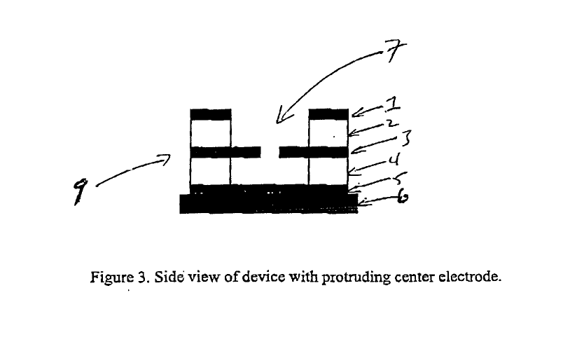

Patterned conductive electrodes 1 constructed on top of the second layer of insulative dielectric material 2 constitute the final layer of each addressable site in the device 9. Well structures 7 are fabricated from this device by conventional photolithography or laser drilling methods used in the semiconductor industry for PCB manufacturing. These wells can have rectangular, circular, trapezoidal or other polygonal openings. Additionally, the well walls may be either straight or curved, and may have an arbitrary angle with respect to the bottom electrode 5. An optional center electrode can alternatively protrude into the well area, as shown in FIG. 3.

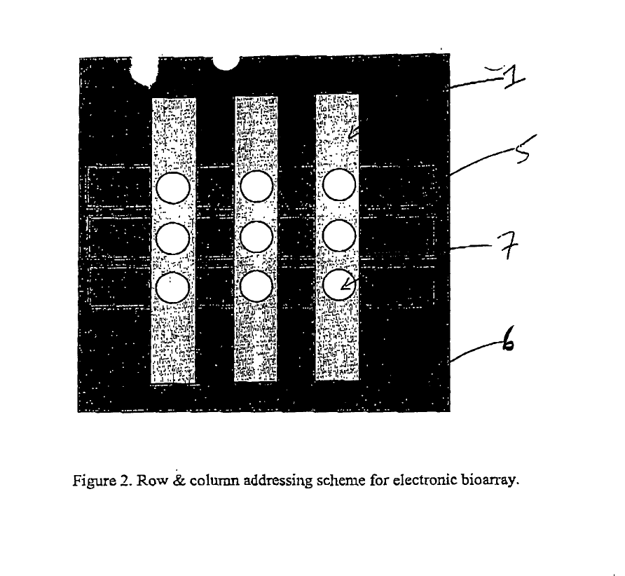

FIG. 2 illustrates a schematic representation of a top view of the apparatus of the invention. The conductive electrodes 1 are preferred to be oriented in a direction orthogonal to the patten1ed conductive electrodes 5, generating row (i.e., patterned electrodes 5) and column (i.e., conductive electrodes 1) addressable high-density electronic or electrochemical mini-cells (i.e., well structures 7) with optional reference electrodes built in-between. The well structure is preferably produced wherein the bottom of the well structure comprises the top of electrode 5 surface, while the top of the well structure is surrounded by the second electrode 1.

The proposed device 9 can be used as an x-y addressable, high-density biochip array when biological probes 10 are immobilized on the patterned electrodes 5 inside each well structure 7. The apparatus is capable of detecting changes in the electrical properties of the probes 10 in each well structure arising from the interaction of the probes 10 with target molecules 11. Though the inventive apparatus is useful for single species detection, where only a few test wells (low density) are required, the advantages of the invention are more pronounced in a high density array where hundreds, thousands, or millions of test wells are integrated in one array.

The following examples serve to more fully describe the manner of using the above-described invention, as well as to set forth the best modes contemplated for carrying out various aspects of the invention. It is understood that these examples in no way serve to limit the true scope of this invention, but rather are presented for illustrative purposes. All references cited herein are incorporated by reference.

### EXAMPLE

### Fabrication of a Linear Microarray With Four Wells

A linear test microarray with four wells was fabricated on a 3″ inch silicon wafer as follows. A photograph of the array is shown in FIG. 4.

The linear test array was fabricated by conventional photolithography in a class 100 clean room and fabrication was performed using three layers of masks as shown in Masks 12 (FIG. 5A), 14 (FIG. 5B) and 16 (FIG. 5C).

A three inch silicon wafer was cleaned using a solution of NH₄OH:H₂O (1:10 v/v), rinsed with de-ionized water, and then dried using a stream of nitrogen at room temperature. On the top of the wafer, 2000Å SiO₂ was deposited by conventional chemical vapor deposition technique.

The array was then prepared sequentially as follows.

1. Bottom Electrode Formation

After cleaning the SiO₂-prepared substrate using a solution of NH₄OH:H₂O (1:10 v/v), a de-ionized water rinse, and drying with a stream of nitrogen as described above, a thick (5-10micron) photoresist (PR) layer was spin-coated on the wafer through a three stage process of spin-coating and softbaking. Using Mask 12 (shown in FIG. 5A) to protect the portion of the substrate that forms the bottom electrode, the surface was exposed to an ultraviolet light source using a wavelength of 365 nm and an intensity of 6 mW/cm³. Following this treatment, the PR was hardbaked and developed. After removal of Mask 12, the following metals were deposited sequentially by evaporation: Ti (to a thickness of 1.0 Angstrom), Au (to a thickness of 21,000 Angstrom), and Ti (to a thickness of 500 Angstrom). After evaporative deposition of these metal layers, a liftoff protocol was used to produce the bottom patterned electrode.

2. Top Electrode Formation

After cleaning the bottom electrode-prepared substrate using a solution of NH₄OH:H₂O (1:10 v/v), a de-ionized water rinse, and drying with a stream of nitrogen as described above, the wafer was coated with a thick (8 micron) layer of PR, as described above. Using Mask 14 (FIG. 5B) to protect the portion of the substrate that forms the top electrode, the surface was exposed to an ultraviolet light source using a wavelength of 365 nm and an intensity of 6 mW/cm³. Following this treatment, the PR was hardbaked and developed as described above. After removal of Mask 14, the following metals were deposited sequentially by evaporation: Ti (to a thickness of 1.0 Angstrom) and Au (to a thickness of 21,000 Angstrom). After evaporative deposition of these metal layers, a liftoff protocol was used to produce the top patterned electrode, as described above.

3. Well Structure Formation

After cleaning the top electrode-prepared substrate using a solution of NH₄OH:H₂O (1:10 v/v), a de-ionized water rinse, and drying with a stream of nitrogen as described above, the wafer was then coated with a 4 micron layer of PR. The surface was exposed to an ultraviolet light source using a wavelength of 365 nm and an intensity of 6 mW/cm³. Following this treatment, the PR was hardbaked and developed as described above. The wafer was then subjected to buffer oxide etching solution (4:1) until each well opening was cleared. The PR was removed by placing in a Branson 4000 Sonicator.

We claim:

1. An apparatus for the detection of target analytes comprising a solid support comprising a grid of electrodes, said support comprising:
a) a first channel comprising a first row of first electrodes each comprising capture binding ligands;
b) at least a second channel comprising a second row of second electrodes comprising capture binding ligands;
wherein said first electrodes share a first row interconnect, said second electrodes share a second row interconnect, and each column of corresponding first and second electrodes share a column interconnect.

2. An apparatus according to claim 1 wherein said first and second channel join to form a feeder channel.

3. An apparatus according to claim 2 wherein said feeder channel comprises a valve.

4. An apparatus according to claim 3 wherein said valve comprises an air bubble.

5. An apparatus according to claim 1 wherein said support further comprises a top to form sealed channels.

6. An apparatus according to claim 1 wherein said support further comprises a hydrophobic layer in between said channels.

7. An apparatus according to claim 1 wherein said apparatus further comprises a voltage source.

8. A method for detecting a target analyte comprising:
a) contacting a sample comprising a target analyte with a apparatus comprising a solid support comprising a grid of electrodes, said support comprising:
i) a first channel comprising a first row of first electrodes each comprising capture binding ligands;
ii) at least a second channel comprising a second row of second electrodes comprising capture binding ligands;
wherein said first electrodes share a first row interconnect, said second electrodes share a second row interconnect, and each column of corresponding first and second electrodes share a column interconnect, under conditions wherein said target analyte will bind at least one of said capture binding ligands; and
b) detecting said target analyte.

9. An apparatus for electrical detection of molecular interactions between immobilized probe molecules and target molecules in a sample solution, comprising:
(a) a supporting substrate comprising an array of test sites;
(b) a set of input electrodes in contact with the supporting substrate, wherein each input electrode is arranged to address a subset of the test sites;
(c) a set of output electrodes in contact with the supporting substrate at the test sites, wherein each output electrode is arranged to address a subset of the test sites, each output electrode is in electrochemical contact with an input electrode, and the output electrodes and input electrodes are intedigitated at the test site;
(d) a plurality of linker moieties in contact with either the input electrodes, the output electrodes, or both the input electrodes and output electrodes at the test sites;
(e) a plurality of probe molecules immobilized to the linker moieties, wherein said probe molecules specifically bind to or interact with target molecules, wherein molecular interactions between the immobilized probe molecules and target molecules are detected as a difference in an electrical signal detected at each output electrode.

10. The apparatus according to claim 9, wherein the supporting substrate comprises ceramic, glass, silicon, silicon nitride, babric, rubber, plastic, printed circuit board, compound semiconductors, or a combination thereof..

11. The apparatus according to claim 9, wherein the input electrodes comprise solid or porous gold, silver, platinum, copper, titanium, chromium, aluminum, metal oxide, metal nitride, metal carbide, carbon, graphite, conductive plastic, metal impregnated polymers, or combinations thereof.

12. The apparatus according to claim 9, wherein the input electrodes comprise a conductive material and an insulating material.

13. The apparatus according to claim 12, wherein the conductive material is selected from the group consisting of gold and platinum.

14. The apparatus according to claim 12, wherein the insulating material is selected from the group consisting of glass, silicon, plastic, rubber, fabric, ceramic, printed circuit board, or combinations thereof.

15. The apparatus according to claim 9, wherein the output electrodes comprise solid or porous gold, silver, platinum, copper, titanium, chromium, aluminum, metal oxide, metal nitride, metal carbide, carbon, graphite, conductive plastic, metal impregnated polymers, or combinations thereof.
